(19) United States
(12) Patent Application Publication
(10) Pub. No.: US 2009/0088461 A1
(43) Pub. Date: Apr. 2, 2009

(54) Methods of Treating or Preventing Cardiac Disease Associated With a High Fat Diet

(75) Inventors: Merlin Thomas, Melbourne (AU); Josephine Forbes, Melbourne (AU); Christos Tikellis, Melbourne (AU)

Correspondence Address: MINTZ, LEVIN, COHN, FERRIS, GLOVSKY AND POPEO, P.C, ONE FINANCIAL CENTER, BOSTON, MA, 02111, US

(73) Assignee: Synvista Therapeutics, Inc., Montvale, NJ (US)

(21) Appl. No.: 12/238,518

(22) Filed: Sep. 26, 2008

Related U.S. Application Data
(60) Provisional application No. 60/995,498, filed on Sep. 26, 2007.

(51) Int. Cl. A61K 31/426; A61P 9/00

(52) U.S. Cl. 514/365

(57) ABSTRACT

The present invention relates to a method of treating or preventing cardiac disorders, myocardial inflammation or myocardial oxidative stress associated with a high fat diet or in a patient subjected to a high fat diet using the thiazolium compounds and compositions of the invention. The present invention also relates to a method of ameliorating weight gain, myocardial AGE accumulation associated, mitochondrial superoxide production, RAGE expression or PPARα expression with a high fat diet or in a patient subjected to a high fat diet using the thiazolium compounds and compositions of the invention.

The present invention claims priority to, and the benefit from, U.S. Patent Application No. 60/995,498, filed Sep. 26, 2007. The contents of this application is incorporated by reference in its entirety.

### BACKGROUND OF THE INVENTION

The classical Western diet, that is high in fat and processing, has a number of adverse effects on cardiovascular physiology. Most research has focused on atherogenesis and vascular stiffness. However, a range of direct cardiac effects may also be observed in the absence of large vessel changes, and prior to the onset of diabetes, including inflammation, hypertrophy¹ (Aguila et al., Mech Ageing Dev. 122:77-88, 2001), fibrosis¹ (Aguila et al., Mech Ageing Dev. 122:77-88, 2001) and contractile dysfunction^{2,3} (Wilson et al., Biochem J. 2007; Ouwens et al., Diabetologia 2007). The mechanisms underlying these cardiac changes are many and varied, and include lipotoxicity^{3,4} (Ouwens et al., Diabetologia 2007; Christoffersen et al., Endocrinology 144:3483-90, 2003), oxidative stress and mitochondrial dysfunction⁵ (Boudina and Abel, Circulation 115:3213-23, 2007). The accumulation of advanced glycation end-products (AGEs) and subsequent activation of the Receptor for AGEs (RAGE) may also represent important mediators of cardiac injury associated with a high fat diet.

A number of pathways may contribute to the accumulation of AGEs following ingestion of a high fat diet. Some AGEs may be formed endogenously due to the induction of dyslipidaemia, dysglycaemia, and oxidative (carbonyl) stress⁶ (Yan et al., Circ Res. 93:1159-69, 2003). However, a significant quantity of AGEs may also be obtained directly from the diet⁷ (Uribarri et al., Ann N Y Acad Sci. 1043:461-6, 2005). In particular, diets rich in fat often have high levels of AGEs, due to chemical interactions between oxidized lipids and protein during high temperature processing⁷ (Uribarri et al., Ann N Y Acad. Sci. 1043:461-6, 2005). Previous studies have shown that increasing the content of AGEs in the diet can increase circulating levels of AGEs. It is also known that circulating levels of AGEs are correlated with cardiac dysfunction⁸ (Berg et al., Diabetes Care 22:1186-90, 1999). Furthermore, inhibition of cardiac AGE accumulation, via a range of different strategies, prevents diabetes- and age-associated increases in myocardial stiffness⁹⁻¹³ (Liu et al., Am J Physiol Heart Circ Physiol. 285:H2587-91, 2003; Ceylan-Isik et al., J Appl Physiol. 100: 150-6, 2006; Corman et al., Proc Natl Acad Sci USA. 95:1301-6, 1998; Candido et al., Circ Res. 92:785-92, 2003; Norton et al., Circulation. 93:1905-12, 1996).

Some of the pathogenic effects of AGEs appear to be mediated by interactions with AGE-receptors⁶ (Yan et al., Circ Res. 93:1159-69, 2003), of which the Receptor for AGEs (RAGE) is the best characterized¹⁴ (Goldin et al., Circulation 114:597-605, 2006). The expression of RAGE is upregulated in diabetes, ageing, and other conditions associated with elevated AGE levels, where it is strongly associated with impaired cardiac function¹⁵ (Simm et al., Exp Gerontol. 39:407-13, 2004). Activation of RAGE is known to influence myocardial calcium homeostasis¹⁶ (Petrova et al., J Mol Cell Cardiol. 2002; 34:1425-31) and contribute to interstitial fibrogenesis in the diabetic heart¹² (Candido et al., Circ Res. 92:785-92, 2003). RAGE is also involved in the activation of inflammatory cascades, including the production of cytokines and chemokines¹⁴ (Goldin et al., Circulation 114:597-605, 2006).

A need exists to identify compounds which modulate the AGE/RAGE axis in cardiac diseases associated with a high fat diet and identify compounds which provide a prophalatic and therapeutic modality for these cardiac disorders.

### SUMMARY OF THE INVENTION

In accordance with the present invention, a method for the prevention or treatment of various conditions or disorders associated with a high fat diet or conditions or disorders in patients subject to a high fat diet using compositions of the invention is provided. In particular, the compositions comprise compounds for inhibiting the formation of and reversing the pre-formed advanced glycosylation (glycation) endproducts and breaking the subsequent cross-links. While not wishing to be bound by any theory, it is believed that the breaking of the pre-formed advanced glycosylation (glycation) endproducts and cross-links is a result of the cleavage of a dicarbonyl-based protein crosslinks present in the advanced glycosylation endproducts. The method and compositions of this invention are thus directed to compounds which, by their ability to effect such cleavage, can be utilized to break the pre-formed advanced glycosylation endproduct and cross-link, and the resultant deleterious effects thereof, both in vitro and in vivo.

The present invention provides methods of treating or preventing a cardiac disorder associated with a high fat diet or in a patient subjected to a high fat diet, by administering to a patient in need thereof, a pharmaceutical composition comprising a thiazolium compound of the present invention and a pharmaceutically acceptable carrier, thereby treating or preventing said disorder. The cardiac disorder can be acute or chronic coronary ischemia, arteriosclerosis, congestive heart failure, angina, atherosclerosis, myocardial hypertrophy, diastolic dysfunction, systolic dysfunction, cardiac hypertrophy, infectious myocarditis, inflammatory myocarditis, chemical myocarditis, cardiomyopathy of any etiology, hypertrophic cardiomyopathy, congenital cardiomyopathy, cardiomyopathy associated with ischemic heart disease or myocardial infarction and or failure.

The present invention also provides methods of treating or preventing myocardial inflammation associated with a high fat diet or in a patient subjected to a high fat diet, by administering to a patient in need thereof, a pharmaceutical composition comprising a thiazolium compound of the present invention and a pharmaceutically acceptable carrier, thereby treating or preventing said inflammation.

In some embodiments, the myocardial inflammation results in increased cardiac expression of macrophage chemo-attractant protein (MCP-1), increased cardiac expression of the intracellular adhesion molecule (ICAM-1), increased cardiac expression of interleukin-6 (IL-6) or increased cardiac expression of tumor necrosis factor alpha (TNFα). The compounds of the present invention reduce or ameliorate the increased cardiac expression of MCP-1, ICAM-1, IL-6 or TNFα associated with a high fat diet or in patients subjected to a high fat diet.

The present invention also provides methods of treating or preventing myocardial oxidative stress associated with a high fat diet or in a patient subjected to a high fat diet, by administering to a patient in need thereof, a pharmaceutical composition comprising a thiazolium compound of the present invention and a pharmaceutically acceptable carrier, thereby treating or preventing said oxidative stress.

In some embodiments, the mitochondrial superoxide production results in increased cardiac expression of the NADPH oxidase subunit gp91^{phox} (NOX-2). The compounds of the present invention reduce or ameliorate the increased cardiac expression of NOX-2 associated with a high fat diet or in patients subjected to a high fat diet.

The present invention also provides methods of ameliorating weight gain associated with a high fat diet or in a patient subjected to a high fat diet, by administering to a patient in need thereof, a pharmaceutical composition comprising a thiazolium compound of the present invention and a pharmaceutically acceptable carrier, thereby ameliorating said weight gain.

The present invention also provides methods of ameliorating myocardial AGE accumulation associated with a high fat diet or in a patient subjected to a high fat diet, by administering to a patient in need thereof, a pharmaceutical composition comprising a thiazolium compound of the present invention and a pharmaceutically acceptable carrier, thereby ameliorating said AGE accumulation.

The present invention also provides methods of ameliorating mitochondrial superoxide production in cardiac cells associated with a high fat diet or in a patient subjected to a high fat diet, by administering to a patient in need thereof, a pharmaceutical composition comprising a thiazolium compound of the present invention and a pharmaceutically acceptable carrier, thereby ameliorating said mitochondrial superoxide production.

The present invention also provides methods of ameliorating RAGE expression or α-type peroxisome proliferator activated receptor (PPARα) expression in cardiac tissue associated with a high fat diet or in a patient subjected to a high fat diet, by administering to a patient in need thereof, a pharmaceutical composition comprising a thiazolium compound of the present invention and a pharmaceutically acceptable carrier, thereby ameliorating said RAGE or PPARα expression.

The present invention provides that a high fat diet derives greater than about 20% of its total calories from fat. In some embodiments, the high fat diet derives greater than about 30% of its total calories from fat. In other embodiments, the high fat diet derives greater than about 40% of its total calories from fat.

The present invention provides that the disorders treated or prevented by the thiazolium compounds of the present invention are not the result of diabetes or adverse sequelae of diabetes, not the result of aging or an age related disorder and not the result of insulin deficiency.

In some embodiments, the present invention provides that the thiazolium compounds of the present invention are administered in combination with a modulator of a receptor for advanced glycation end-products (RAGE). The compounds and modulator can be administered simultaneously or sequentially, in any order.

The invention comprises thiazolium compounds having the following structural formula:

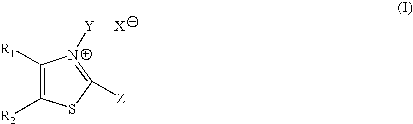

wherein R¹ is selected from the group consisting of hydrogen, hydroxy (lower) alkyl, acetoxy (lower) alkyl, lower alkyl, lower alkenyl;
R² is selected from the group consisting of hydrogen, hydroxy (lower) alkyl, acetoxy (lower) alkyl, lower alkyl, lower alkenyl; or R¹ and R² together with their ring carbons may be an aromatic fused ring, optionally substituted by one or more amino, halo or alkylenedioxy groups;
Z is hydrogen or an amino group;
Y is amino, a group of the formula:

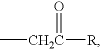

wherein R is a lower alkyl, alkoxy, hydroxy, amino or an aryl group, said aryl group optionally substituted by one or more lower alkyl, lower alkoxy, halo, dialkylamino, hydroxy, nitro or alkylenedioxy groups;
a group of the formula:

—CH₂R′

wherein R′ is hydrogen, or a lower alkyl, lower alkenyl, or aryl group; or a group of the formula:

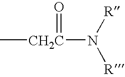

wherein R″ is hydrogen and R″ is a lower alkyl group, optionally substituted by an aryl group, or an aryl group, said aryl group optionally substituted by one or more lower alkyl, halo, or alkoxylcarbonyl groups; or R″ and R′″ are both lower alkyl groups;
X is a halide, tosylate, methanesulfonate or mesitylenesulfonate ion;
and mixtures thereof, and a carrier therefor.

The preferred thiazolium compound of the instant invention comprises the structure of Formula I, wherein R¹ and R² are lower alkyl, Z is hydrogen, Y is a group of the formula

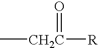

wherein R is an aryl group and X is halide. In more preferred embodiments, the compound of the invention is 3-(2-phenyl-2-oxoethyl)-4,5-dimethylthiazolium chloride or N-phenacyl-4,5-dimethylthiazolium chloride, also referred to as ALT-711 or alagebrium chloride herein. In other embodiments, the compound of the invention is 3-(2-phenyl-2-oxoethyl)-4,5-dimethylthiazolium bromide or N-phenacyl-4,5-dimethylthiazolium bromide, also referred to as DMPTB or PMTB.

The compounds, and their compositions, utilized in this invention appear to react with an early glycosylation product thereby preventing the same from later forming the advanced glycosylation end products which lead to cross-links, and thereby, to molecular or protein aging and other adverse molecular consequences. Additionally, they react with already formed advanced glycosylation end products to reduce the amount of such products.

The invention further extends to the identification and use of a novel cross-link structure which is believed to represent a significant number of the molecular crosslinks that form in vitro and in vivo as a consequence of advanced glycation. More particularly, the cross-link structure includes a sugar-derived α-dicarbonyl segment or moiety, such as a diketone, that is capable of cleavage by a dinucleophilic, thiazolium-like compound.

Specifically, the cross-link structure may be according to the formula:

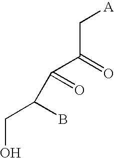

where A and B independently, are sites of attachment to the nucleophilic atom of a biomolecule.

Accordingly, it is a principal object of the present invention to provide a method for the treatment or prevention of cardiac disorders associated with a high fat diet or in patients with a high fat diet, where the formation of advanced glycosylation endproducts and extensive cross-linking of molecules is inhibited, and cross-links formed from pre-existing advanced glycosylation endproducts, that occur as a consequence of the reaction of susceptible molecules such as proteins with glucose and other reactive sugars, by correspondingly inhibiting the formation of advanced glycosylation endproducts, are broken and the breaking of the advanced glycosylation mediated cross-linking has previously occurred. Preferably, the cardiac disorder is treated or prevented by administering a compound of the invention to a subject in need thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description.

### BRIEF DESCRIPTION OF THE DRAWINGS

FIG. 1 is a photograph of immunohistochemistry staining showing that a HF diet was associated with increased accumulation of interstitial collagen as demonstrated by picrosirus staining (left column), increased perivascular collagen as demonstrated on Van-Giesen stained sections (second column) and expression of collagen IV (third column). Collagen accumulation associated with high fat feeding was reduced in RAGE KO mice (HF+KO, bottom row), in mice receiving alegebrium chloride (HF+AL, third row) Cardiac fat accumulation was increased in fat fed mice as demonstrated by staining with oil red-o (right column), and this increase was unaffected in RAGE KO mice and mice receiving alegebrium chloride.

FIG. 2 is a graph showing the expression of IL-6 protein in LV tissues.

FIG. 3 is a graph showing the accumulation of AGEs and the RAGE ligand, S100 A8/A9 in LV tissues.

FIG. 4 is a graph showing superoxide production in LV homogenates.

### DETAILED DESCRIPTION

AGEs represent important mediators of cardiac injury associated with a high fat diet. The present invention provides that inhibition of AGE accumulation following treatment with thiazolium compounds, such as alagebrium chloride, and prevention of RAGE activation are able to prevent the induction of inflammation, oxidative stress and mitochondrial dysfunction in the heart associated with high fat feeding.

The present invention provides methods of treating or preventing a cardiac disorder associated with a high fat diet or in a patient subjected to a high fat diet, by administering to a patient in need thereof, a pharmaceutical composition comprising a thiazolium compound of Formula I and a pharmaceutically acceptable carrier, thereby treating or preventing said disorder.

The cardiac disorder can include, but is not limited to, acute or chronic coronary ischemia, arteriosclerosis, congestive heart failure, angina, atherosclerosis, myocardial hypertrophy, diastolic dysfunction, systolic dysfunction, cardiac hypertrophy, infectious myocarditis, inflammatory myocarditis, chemical myocarditis, cardiomyopathy of any etiology, hypertrophic cardiomyopathy, congenital cardiomyopathy, cardiomyopathy associated with ischemic heart disease or myocardial infarction and or failure.

The present invention also provides methods of treating or preventing myocardial inflammation associated with a high fat diet or in a patient subjected to a high fat diet, by administering to a patient in need thereof, a pharmaceutical composition comprising a thiazolium compound of Formula I and a pharmaceutically acceptable carrier, thereby treating or preventing said inflammation. In some embodiments, the myocardial inflammation results in increased cardiac expression of macrophage chemo-attractant protein (MCP-1) or increased cardiac expression of the intracellular adhesion molecule (ICAM-1). The compounds of the present invention reduce or ameliorate the increased cardiac expression of MCP-1, ICAM-1, IL-6 or TNFα associated with a high fat diet or in patients subjected to a high fat diet.

The present invention also provides methods of treating or preventing myocardial oxidative stress associated with a high fat diet or in a patient subjected to a high fat diet, by administering to a patient in need thereof, a pharmaceutical composition comprising a thiazolium compound of Formula I and a pharmaceutically acceptable carrier, thereby treating or preventing said oxidative stress. In some embodiments, the mitochondrial superoxide production results in increased cardiac expression of the NADPH oxidase subunit gp91^{phox}(NOX-2). The compounds of the present invention reduce or ameliorate the increased cardiac expression of NOX-2 associated with a high fat diet or in patients subjected to a high fat diet.

The present invention also provides methods of ameliorating weight gain associated with a high fat diet or in a patient subjected to a high fat diet, by administering to a patient in need thereof, a pharmaceutical composition comprising a thiazolium compound of Formula I and a pharmaceutically acceptable carrier, thereby ameliorating said weight gain.

The present invention also provides methods of ameliorating myocardial AGE accumulation associated with a high fat diet or in a patient subjected to a high fat diet, by administering to a patient in need thereof, a pharmaceutical composition comprising a thiazolium compound of Formula I and a pharmaceutically acceptable carrier, thereby ameliorating said AGE accumulation.

The present invention also provides methods of ameliorating mitochondrial superoxide production in cardiac cells associated with a high fat diet or in a patient subjected to a high fat diet, by administering to a patient in need thereof, a pharmaceutical composition comprising a thiazolium compound of Formula I and a pharmaceutically acceptable carrier, thereby ameliorating said mitochondrial superoxide production.

The present invention also provides methods of ameliorating RAGE expression or α-type peroxisome proliferator activated receptor (PPARα) expression in cardiac tissue associated with a high fat diet or in a patient subjected to a high fat diet, by administering to a patient in need thereof, a pharmaceutical composition comprising a thiazolium compound of Formula I and a pharmaceutically acceptable carrier, thereby ameliorating said RAGE or PPARα expression.

The present invention provides that a high fat diet derives greater than about 20% of its total calories from fat. In some embodiments, the high fat diet derives greater than about 30% of its total calories from fat. In other embodiments, the high fat diet derives greater than about 40% of its total calories from fat.

The present invention provides that the disorders treated or prevented by the thiazolium compounds of Formula I are not the result of diabetes or adverse sequelae of diabetes, not the result of aging or an age related disorder and not the result of insulin deficiency.

In some embodiments, the present invention provides that the thiazolium compounds of Formula I are administered in combination with a modulator of a receptor for advanced glycation end-products (RAGE). The compounds Formula I and RAGE modulator can be administered simultaneously or sequentially, in any order.

The thiazolium compounds for use in the present invention comprise the compounds of Formula I,

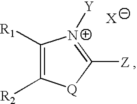

wherein:
R¹ and R² are independently selected from hydrogen, C₁₋₆ linear or branched alkyl and cycloalkyl; or together with their ring carbons form a C₅-C₇ fused cycloalkyl ring having up to two double bonds including any fused double bond of the -olium containing ring, which cycloalkyl ring is optionally substituted by one or more substituents selected from alkyl and fluoro;
Z is hydrogen or C₁₋₆ linear or branched alkyl;
Y is a group of the formula —CH(R⁵)—C(O)—R⁶ wherein
R⁵ is hydrogen, C₁₋₆ linear- or branched-alkyl, or cycloalkyl; and
R⁶ is a C₆ or C₁₀ aryl, wherein R⁶ is optionally substituted with one or more substituents selected from the group consisting of alkyl and halo;

### Q is O or S; and

X is a pharmaceutically acceptable anion.

In some embodiments, R1 and R2 are independently C₁₋₆ linear or branched alkyl. In some embodiments, Z is hydrogen. In some embodiments, R⁵ is hydrogen. In some embodiments, R⁶ is C₆ aryl. In some embodiments, Q is S.

In preferred embodiments the compound of Formula I is 3-(2-phenyl-2-oxoethyl)-4,5-dimethylthiazolium. In more preferred embodiments, the compound of Formula I is 3-(2-phenyl-2-oxoethyl)-4,5-dimethylthiazolium chloride or 3-(2-phenyl-2-oxoethyl)-4,5-dimethylthiazolium bromide.

In accordance with the present invention, compounds, compositions including pharmaceutical compositions containing said compounds and associated methods have been developed to inhibit the formation of advanced glycosylation endproducts in a number of target molecules, including particularly proteins, existing in both animals and plant material, and to reverse the already formed advanced glycosylation endproducts. In particular, the invention relates to a composition which may contain one or more compounds having the ability to effect cleavage of α-dicarbonyl-based molecular crosslinks present in the advanced glycosylation endproducts. In particular, the invention relates to compositions that can reverse the accumulation of AGEs and reduction of AGE-associated cardiac disorders which occurs in patients subject to a high fat diet.

Additional useful compounds, for instance, comprise compounds having the structural formula:

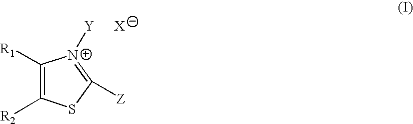

wherein R¹ and R² are independently selected from the group consisting of hydrogen, hydroxy(lower)alkyl, acetoxy(lower)alkyl, lower alkyl, lower alkenyl, or R¹ and R² together with their ring carbons may be an aromatic fused ring, optionally substituted by one or more amino, halo or alkylenedioxy groups;
Z is hydrogen or an amino group;
Y is amino, a group of the formula

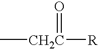

wherein R is a lower alkyl, alkoxy, hydroxy, amino or an aryl group, said aryl group optionally substituted by one or more lower alkyl, lower alkoxy, halo, dialkylamino, hydroxy, nitro or alkylenedioxy groups;
a group of the formula

—CH₂R′

wherein R′ is hydrogen, or a lower alkyl, lower alkynyl, or aryl group; or a group of the formula:

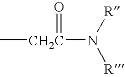

wherein R″ is hydrogen and R′″ is a lower alkyl group, optionally substituted by an aryl group, or an aryl group, said aryl group optionally substituted by one or more lower alkyl, halo, or alkoxylcarbonyl groups; or R″ and R′″ are both lower alkyl groups;
X is a halide, tosylate, methanesulfonate or mesitylenesulfonate ion; and mixtures thereof, and a carrier therefor.

The term “lower alkyl” means that the group contains 1, 2, 3, 4, 5, or 6 carbon atoms and includes methyl, ethyl, propyl, butyl, pentyl, hexyl, and the corresponding branched-chain isomers thereof. The term “lower alkynyl” means that the group contains from 2, 3, 4, 5, or 6 carbon atoms. Similarly, the term “lower alkoxy” means that the group contains from 1, 2, 3, 4, 5, or 6 carbon atoms, and includes methoxy, ethoxy, propoxy, butoxy, pentoxy, and hexoxy, and the corresponding branched-chain isomers thereof. These groups are optionally substituted by one or more halo, hydroxy, amino or lower alkylamino groups.

The term “lower acyloxy(lower)alkyl” means that the acyloxy portion contain from 2, 3, 4, 5, or 6 carbon atoms and the lower alkyl portion contains from 1, 2, 3, 4, 5, or 6 carbon atoms. Typical acyloxy portions are those such as acetoxy or ethanoyloxy, propanoyloxy, butanoyloxy, pentanoyloxy, hexanoyloxy, and the corresponding branched chain isomers thereof. Typical lower alkyl portions are as described hereinabove.

The aryl groups encompassed by the formulae of the invention are those containing 6, 7, 8, 9, or 10 carbon atoms, such as naphthyl, phenyl and lower alkyl substituted-phenyl, e.g., tolyl and xylyl, and are optionally substituted by 1-2 halo, hydroxy, lower alkoxy or di (lower) alkylamino groups. Preferred aryl groups are phenyl, methoxyphenyl and 4-bromophenyl groups.

The halo atoms in the formulae of the invention may be fluoro, chloro, bromo or iodo. For the purposes of this invention, the compounds of the invention are formed as biologically and pharmaceutically acceptable salts. Useful salt forms are the halides, particularly the bromide and chloride, tosylate, methanesulfonate, and mesitylenesulfonate salts. Other related salts can be formed using similarly non-toxic, and biologically and pharmaceutically acceptable anions.

The preferred thiazolium compound of the instant invention comprises the structure of Formula I, wherein R¹ and R² are lower alkyl, Z is hydrogen, Y is a group of the formula

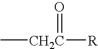

wherein R is an aryl group and X is halide. In more preferred embodiments, the compound of the invention is 3-(2-phenyl-2-oxoethyl)-4,5-dimethylthiazolium chloride or N-phenacyl-4,5-dimethylthiazolium chloride, also referred to as ALT-711 or alagebrium chloride herein. In other embodiments, the compound of the invention is 3-(2-phenyl-2-oxoethyl)-4,5-dimethylthiazolium bromide or N-phenacyl-4,5-dimethylthiazolium bromide, also referred to as DMPTB or PMTB.

As used herein, “treating” or “treatment” includes any effect e.g., lessening, reducing, modulating, or eliminating, that results in the improvement of the condition, disease, disorder, etc. “Treating” or “treatment” of a disease state means the treatment of a disease-state in a mammal, particularly in a human, and include: (a) inhibiting an existing disease-state, i.e., arresting its development or its clinical symptoms; and/or (c) relieving the disease-state, i.e., causing regression of the disease state.

As used herein, “preventing” means causing the clinical symptoms of the disease state not to develop i.e., inhibiting the onset of disease, in a subject that may be exposed to or predisposed to the disease state, but does not yet experience or display symptoms of the disease state.

As used herein, “ameliorating” means includes any effect e.g., lessening, reducing, modulating, or eliminating, that results in the improvement of the condition, disease, disorder, etc.

As used herein, “high fat diet” or “western diet” means a dietary consumption which contains greater than 20% of its total calories from fat. In some embodiments, the dietary consumption contains greater than 30% of its total calories from fat. In other embodiments, the dietary consumption contains greater than 40% of its total calories from fat. “High fat diet” or “western diet” are used interchangeably herein. These figures are based on guidelines by the Food and Drug Administration and the Food Safety and Inspection Service of the U.S. Department of Agriculture.

As used herein, “subjected to a high fat diet” means subjects or patients which consume a high fat diet as described above.

Of the compounds encompassed by Formula I, certain substituents are preferred. For instance, the compounds wherein R¹ or R² are lower alkyl groups are preferred. Also highly preferred are the compounds wherein Y is an amino group, a 2-amino-2-oxoethyl group, a 2-phenyl-2-oxoethyl or a 2-(substituted phenyl)-2-oxoethyl group.

Representative compounds of the present invention are:
3-aminothiazolium mesitylenesulfonate;
3-amino-4,5-dimethylaminothiazolium mesitylenesulfonate;
2,3-diaminothiazolinium mesitylenesulfonate;
3-(2-methoxy-2-oxoethyl)-thiazolium bromide;
3-(2-methoxy-2-oxoethyl)-4,5-dimethylthiazolium bromide;
3-(2-methoxy-2-oxoethyl)-4-methylthiazolium bromide;
3-(2-phenyl-2-oxoethyl)-4-methylthiazolium bromide;
3-(2-phenyl-2-oxoethyl)-4,5-dimethylthiazolium bromide;
3-(2-phenyl-2-oxoethyl)-4,5-dimethylthiazolium chloride;
3-amino-4-methylthiazolium mesitylenesulfonate;
3-(2-methoxy-2-oxoethyl)-5-methylthiazolium bromide;
3-(3-(2-phenyl-2-oxoethyl)-5-methylthiazolium bromide;
3-[2-(4′-bromophenyl)-2-oxoethyl]thiazolium bromide;
3-[2-(4′-bromophenyl)-2-oxoethyl]-4-methylth′iazolium bromide;
3-[2-(4′-bromophenylDhenyl)-2-oxoethyl]-5-methylthiazolium bromide;
3-[2-(4′bromophenyl)-2-oxoethyl)-4,5-dimethylthiazolium bromide;
3-(2-methoxy-2-oxoethyl)-4-methyl-5-(2-hydroxyethyl)thiazolium bromide;
3-(2-phenyl-2-oxoethyl)-4-methyl-5-(2-hydroxyethyl)thiazolium bromide;
3-[2-(4′-bromophenyl)-2-oxoethyl]-4-methyl-5-(2-hydroxyethyl)thiazolium bromide;
3,4-dimethyl-5-(2-hydroxyethyl)thiazolium iodide;
3-ethyl-5-(2-hydroxyethyl)-4-methylthiazolium bromide;
3-benzyl-5-(2-hydroxyethyl)-4-methylthiazolium chloride;
3-(2-methoxy-2-oxoethyl)benzothiazolium bromide;
3-(2-phenyl-2-oxoethyl)benzothiazolium bromide;
3-[2-(4′bromophenyl)-2-oxoethyl]benzothiazolium bromide;
3-(carboxymethyl)benzothiazolium bromide;
2,3-(diamino)benzothiazolium mesitylenesulfonate;
3-(2-amino-2-oxoethyl)thiazolium bromide;
3-(2-amino-2-oxoethyl)-4-methylthiazolium bromide;
3-(2-amino-2-oxoethyl)-5-methylthiazolium bromide;
3-(2-amino-2-oxoethyl)4,5-dimethylthiazolium bromide;
3-(2-amino-2-oxoethyl)benzothiazolium bromide;
3-(2-amino-2-oxoethyl)4-methyl-5-(2-hydroxyethyl)thiazolium bromide;
3-amino-5-(2-hydroxyethyl)-4-methylthiazolium mesitylenesulfonate;
3-(2-methyl-2-oxoethyl)thiazolium chloride;
3-amino-4-methyl-5-(2-acetoxyethyl)thiazolium mesitylenesulfonate;
3-(2-phenyl-2-oxoethyl)thiazolium bromide;
3-(2-methoxy-2-oxoethyl)-4-methyl-5-(2-acetoxyethyl)thiazolium bromide;
3-(2-amino-2-oxoethyl)-4-methyl-5-(2-acetoxyethyl)thiazolium bromide;
2-amino-3-(2-methoxy-2-oxoethyl)thiazolium bromide;
2-amino-3-(2-methoxy-2-oxoethyl)benzothiazolium bromide;
2-amino-3-(2-amino-2-oxoethyl)thiazolium bromide;
2-amino-3-(2-amino-2-oxoethyl)benzothiazolium bromide;
3-[2-(4′-methoxyphenyl)-2-oxoethyl)-thiazolinium bromide;
3-[2-(2′,4′-dimethoxyphenyl)-2-oxoethyl]-thiazolinium bromide;
3-[2-(4′-fluorophenyl)-2-oxoethyl]-thiazolinium bromide;
3-[2-(2′,4′-difluorophenyl)-2-oxoethyl]-thiazolinium bromide;
3-[2-(4′-diethylaminophenyl)-2-oxoethyl]-thiazolinium bromide;
3-propargyl-thiazolinium bromide;
3-propargyl-4-methylthiazolinium bromide;
3-propargyl-5-methylthiazolinium bromide;
3-propargyl-4,5-dimethylthiazolinium bromide;
3-propargyl-4-methyl-5-(2-hydroxyethyl)-thiazolinium bromide;
3-(2-(3′-methoxyphenyl)-2-oxoethyl)-thiazolium bromide;
3-(2-(3′-methoxy phenyl)-2-oxoethyl)-4 methyl-5-(2′-hydroxyethyl)-thiazolium bromide;
3-(2-(3′-methoxyphenyl)-2-oxoethyl)-benzothiazolium bromide;
2,3-diamino-4-chlorobenzothiazolium mesitylenesulfonate;
2,3-diamino-4-methyl-thiazolium mesitylenesulfonate;
3-amino-4-methyl-5-vinyl-thiazolium mesitylenesulfonate;
2,3-diamino-6-chlorobenzothiazolium mesitylenesulfonate;
2,6-diamino-benzothiazole dihydrochloride;
2,6-diamino-3 [2-(4′-methoxyphenyl)-2-oxoethyl)benzothiazolium bromide;
2,6-diamino-3 [2-(3′-methoxyphenyl)-2-oxoethyl)benzothiazolium bromide;
2,6-diamino-3 [2-(4′-diethylaminophenyl)-2-oxoethyl]benzothiazolium bromide;
2,6-diamino-3 (2-(4′-bromophenyl)-2-oxoethyl]benzothiazolium bromide;
2,6-diamino-3 (2-(2-phenyl-2-oxoethyl)benzothiazolium bromide;
2,6-diamino-3 [2-(4′-fluorophenyl-2-oxoethyl)benzothiazolium bromide;
3-acetamido-4-methyl-5-thiazolyl-ethyl acetate mesitylenesulfonate;
2,3-diamino-5-methylthiazolium mesitylenesulfonate;
3-[2-(2′-naphtyl)-2-oxoethyl]-4-methyl-5-(2′-hydroxyethyl)-thiazolium bromide;
3-[2-(3′,5′-di-ter-butyl-4′-hydroxyphenyl)-2-oxoethyl]-4-methyl-5-(2′-hydroxyethyl-thiazolium bromide;
3-[2-(2′,6′-dichlorophenethylamino)-2-oxoethyl]-4-methyl-5-(2′-hydroxyethyl)-thiazolium-bromide;
3-[2-dibutylamino-2-oxoethyl)-4-methyl-5-(2′-hydroxyethyl)-thiazolium bromide;
3-[2-4′-carbethoxyanilino)-2-oxoethyl]-4-methyl-5-(2′-hydroxyethyl)-thiazolium bromide;
3-[2-(2′,6′-diisopropylanilino)-2-oxoethyl]-4-methyl-5-(2′-hydroxyethyl)-thiazolium bromide;
3-amino-4-methyl-5-(2-(2′,6′-dichlorobenzyloxy)ethyl]-thiazolium mesitylenesulfonate;
3-[2-(4′-carbmethoxy-3′-hydroxyanilino)-2-oxoethyl)-4-methyl-5-(2′-hydroxyethyl)-thiazolium bromide;
2,3-diamino-4,5-dimethylthiazolium mesitylenesulfonate;
2,3-diamino-4-methyl-5-hydroxyethyl-thiazolium mesitylene sulfonate;
2,3-diamino-5-(3′,4′-trimethylenedioxy phenyl)-thiazolium mesitylene sulfonate;
3-[2-(1′,4′-benzodioxan-6-yl)-2-oxoethyl]-4-methyl-5-(2′-hydroxyethyl)-thiazolium bromide;
3-[2-(3′,4′-trimethylenedioxyphenyl)-2-oxoethyl]-4-methyl-5-(2′-hydroxyethyl)-thiazolium bromide;
3-(2-[1′,4-benzodioxan-6-yl)-2-oxoethyl)-thiazolium bromide;
3-[2-(3′,4′-trimethylenedioxyphenyl)-2-oxoethyl]-thiazolium bromide;
3-[2-(3′,5′-di-tert-butyl-4′-hydroxyphenyl)-2-oxoethyl]-thiazolium bromide;
3-[2-(3′,5′-di-tert-butyl-4′-hydroxyphenyl)-2-oxoethyl]-4-methyl-thiazolium bromide;
3-[2-(3′,5′-di-tert-butyl-4′-hydroxyphenyl)-2-oxoethyl)-5-methyl-thiazolium bromide;
3-[2-(3′,5′-di-tert-butyl-4′-hydroxyphenyl)-2-oxoethyl]-4,5-dimethyl-thiazolium bromide;
3-[2-(3′,5′-di-tert-butyl-4′-hydroxyphenyl)-2-oxoethyl]-benzothiazolium bromide;
1-methyl-3-(2-(3′,5′-di-tert-butyl-4′-hydroxyphenyl)-2-oxoethyl]-imidazolium bromide;
3-[2-(4′-n-pentylphenyl)-2-oxoethyl]-thiazolinium bromide;
3-[2-(4′-n-pentylphenyl)-2-oxoethyl]-4-methyl-5-(2′-hydroxyethyl)-thiazolinium bromide;
3-[2-4′-diethylaminophenyl)-2-oxoethyl]-4-methyl-5-(2′-hydroxyethyl)-thiazolinium bromide;
3-(2-phenyl-2-oxoethyl)-4-methyl-5-vinyl-thiazolium bromide;
3-[2-(3′,5′-tert-butyl-4′-hydroxyphenyl)-2-oxoethyl)-4-methyl-5-vinyl-thiazolium bromide;
3-(2-tert-butyl-2-oxoethyl)-thiazolium bromide;
3-(2-tert-butyl-2-oxoethyl)-4-methyl-5-(2′-hydroxyethyl)-thiazolium bromide;
3-(3′-methoxybenzyl)-4-methyl-5-(2′-hydroxyethyl)-thiazolium chloride;
3-(2′,6′-dichlorobenzyl)-4-methyl-5-(2′-hydroxyethyl)-thiazolium chloride;
3-(2′-nitrobenzyl)-4-methyl-5-(2′-hydroxyethyl)-thiazolium bromide;
3[2-(4′-chlorophenyl)-2-oxoethyl]-thiazolium bromide;
3[2-(4′-chlorophenyl)-2-oxoethyl]-4-methyl-5-(2′-hydroxyethyl)-thiazolium bromide; and
3[2-(4′-methoxyphenyl)-2-oxoethyl]-4-methyl-5-(2′-hydroxyethyl)-thiazolium bromide.

Compounds of the invention further include compounds represented by the formula Ia:

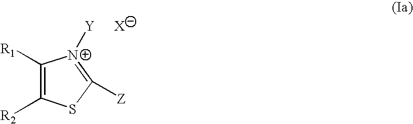

wherein R¹ is independently selected from the group consisting of hydrogen, hydroxy(lower)alkyl, acetoxy(lower)alkyl, lower alkyl, lower alkenyl;
R² is independently selected from the group consisting of hydrogen, hydroxy(lower)alkyl, acetoxy(lower)alkyl, lower alkyl, lower alkenyl, or R¹ and R² together with their ring carbons may be an aromatic fused ring, optionally substituted by one or more amino, halo or alkylenedioxy groups;
Z is hydrogen or an amino group;
Y is amino, a group of the formula

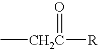

wherein R is a lower alkyl, alkoxy, hydroxy, amino or an aryl group, said aryl group optionally substituted by one or more lower alkyl, lower alkoxy, halo, dialkylamino, hydroxy, nitro or alkylenedioxy groups;
a group of the formula

—CH₂R′

wherein R′ is hydrogen, or a ‘lower alkyl, lower alkynyl, or aryl group; or a group of the formula

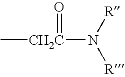

wherein R″ is hydrogen and R′″ is a lower alkyl group, optionally substituted by an aryl group, or an aryl group, said aryl group optionally substituted by one or more lower alkyl, halo, or alkoxylcarbonyl groups; or R″ and R″ are both lower alkyl groups;
with the proviso that at least one of Y and Z is an amino group, and the further proviso that when Y is amino and R² and Z are both hydrogen, then R¹ is other than a lower alkyl group; and
X is a halide, tosylate, methanesulfonate or mesitylenesulfonate ion.

The preferred thiazolium compound of the instant invention comprises the structure of Formula I, wherein R¹ and R² are lower alkyl, Z is hydrogen, Y is a group of the formula

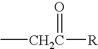

wherein R is an aryl group and X is halide. In more preferred embodiments, the compound of the invention is 3-(2-phenyl-2-oxoethyl)-4,5-dimethylthiazolium chloride or N-phenacyl-4,5-dimethylthiazolium chloride, also referred to as ALT-711 or alagebrium chloride herein. In other embodiments, the compound of the invention is 3-(2-phenyl-2-oxoethyl)-4,5-dimethylthiazolium bromide or N-phenacyl-4,5-dimethylthiazolium bromide, also referred to as DMPTB or PMTB.

Other compounds of the invention are those of the formula Ib:

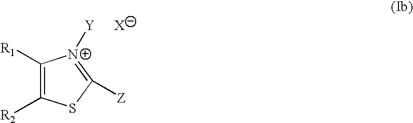

wherein R¹ is independently selected from the group consisting of, hydroxy (lower) alkyl, acetoxy(lower)alkyl, lower acyloxy(lower)alkyl, lower alkyl;
R² is independently selected from the group consisting of, hydroxy (lower) alkyl, acetoxy(lower)alkyl, lower acyloxy(lower)alkyl, lower alkyl, or R¹ and R² together with their ring carbons may be an aromatic fused ring;
Z is hydrogen or an amino group;
Y is an alkynylmethyl group, or a group of the formula

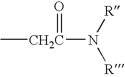

wherein R″ is hydrogen and R″ is a lower alkyl group, optionally substituted by an aryl group, or an aryl group, said aryl group optionally substituted by one or more lower alkyl, halo, or alkoxylcarbonyl groups; or R″ and R′″ are both lower alkyl groups; and X is a halide, tosylate, methanesulfonate or mesitylenesulfonate ion.

Other compounds of the invention are those of formula (Ic):

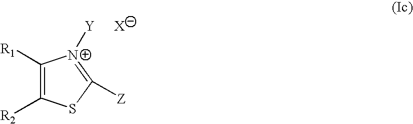

wherein R¹ and R² are methyl; Z is hydrogen;
Y is a group of the formula:

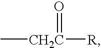

wherein R is phenyl; and
X is halide.

The above compounds are capable of inhibiting the formation of advanced glycosylation endproducts on target molecules, including, for instance, proteins, as well as being capable of breaking or reversing already formed advanced glycosylation endproducts on such proteins. The compounds employed in accordance with this invention inhibit this late-stage Maillard effect and reduce the level of the advanced glycosylation endproducts already present in the protein material.

The rationale of the present invention is to use compounds which block, as well as reverse, the post-glycosylation step, e.g., the formation of fluorescent chromophores and cross-links, the presence of which is associated with a high fat diet, and leads to cardiac dysfunction and disease. An ideal agent would prevent the formation of such chromophores and of cross-links between protein strands and trapping of proteins onto other proteins, such as occurs in the heart and cardiac tissue, and reverse the level of such cross-link formation already present.

The chemical nature of the early glycosylation products with which the compounds of the present invention are believed to react may vary, and accordingly the term “early glycosylation product(s)” as used herein is intended to include any and all such variations within its scope. For example, early glycosylation products with carbonyl moieties that are involved in the formation of advanced glycosylation endproducts, and that may be blocked by reaction. with the compounds of the present invention, have been postulated. In one embodiment, it is envisioned that the early glycosylation product may comprise the reactive carbonyl moieties of Amadori products or their further condensation, dehydration and/or rearrangement products, which may condense to form advanced glycosylation endproducts. In another scenario, reactive carbonyl compounds, containing one or more carbonyl moieties (such as glycolaldehyde, glyceraldehyde or 3-deoxyglucosone) may form from the cleavage of Amadori or other early glycosylation endproducts, and by subsequent reactions with an amine or Amadori product, may form carbonyl containing advanced glycosylation products such as alkylformyl-glycosylpyrroles.

Several investigators have studied the mechanism of advanced glycosylation product formation. In vitro studies by Eble et al., (1983), “Nonenzymatic Glucosylation and Glucose-dependent Cross-linking of Protein”, J. Biol. Chem., 258:9406-9412, concerned the cross-linking of glycosylated protein with nonglycosylated protein in the absence of glucose. Eble et al. sought to elucidate the mechanism of the Maillard reaction and accordingly conducted controlled initial glycosylation of RNase as a model system, which was then examined under ‘varying conditions. In one aspect, the glycosylated protein material was isolated and placed in a glucose-free environment and thereby observed to determine the extent of cross-linking.

Eble et al. thereby observed that cross-linking continued to occur not only with the glycosylated protein but with non-glycosylated proteins as well. One of the observations noted by Eble et al. was that the reaction between glycosylated protein and the protein material appeared to occur at the location on the amino acid side chain of the protein. Confirmatory experimentation conducted by Eble et al. in this connection demonstrated that free lysine would compete with the lysine on RNase for the binding of glycosylated protein. Thus, it might be inferred from these data that lysine may serve as an inhibitor of advanced glycosylation; however, this conclusion and the underlying observations leading to it should be taken in the relatively limited context of the model system prepared and examined by Eble et al. Clearly, Eble et al. does not appreciate, nor is there a suggestion therein, of the discoveries that underlie the present invention, with respect to the inhibition of advanced glycosylation of proteins both in vitro and in vivo.

While not wishing to be bound by any particular theory as to the mechanism by which the compounds of the instant invention reverse already formed advanced glycosylation endproducts, studies have been structured to elucidate a possible mechanism. Earlier studies examining the fate of the Amadori product (AP) in vivo have identified one likely route that could lead to the formation of covalent, glucose-derived protein crosslinks. This pathway proceeds by dehydration of the AP via successive beta-eliminations as shown in the Scheme A below. Thus, loss of the 4-hydroxyl of the AP (1) gives a 1,4-dideoxy-1-alkylamino-2,3-hexodiulose (AP-dione) (2). An AP-dione with the structure of an amino-1,4-dideoxyosone has been isolated by trapping model APs with the AGE-inhibitor aminoguanidine. Subsequent elimination of the 5-hydroxyl gives a 1,4,5-trideoxy-1-alkylamino-2,3-hexulos-4-ene (AP-ene-dione) (3), which has been isolated as a triacetyl derivative of its 1,2-enol form. Amadori-diones, particularly the AP-ene-dione, would be expected to be highly reactive toward protein cross linking reactions by serving as targets for the addition of the amine (Lys, His)-, or sulfhydryl (Cys)-based nucleophiles that exist in proteins, thereby producing stable cross links of the form (4).

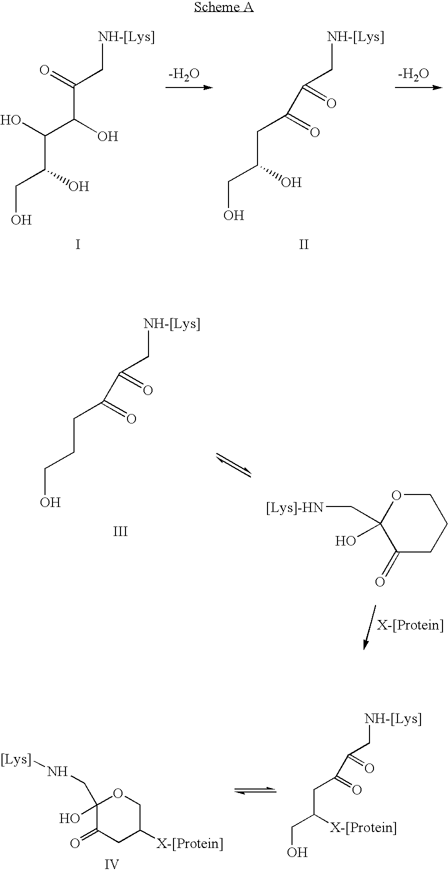

Note that the linear AP-ene-dione of (3) and the stable 20 cross-link of, (4) may cyclize to form either 5- or 6-member lactol rings, although only the 6-member cyclic variant is shown in Scheme A set forth above.

The possibility that a major pathway of cross link formation proceeds through an AP-ene-dione intermediate was investigated by experiments designed to test the occurrence of this pathway in vivo as well as to effect the specific cleavage of the resultant α-dicarbonyl-based protein crosslinks. The thiazolium compounds of the instant invention are thus believed to act as novel “bidentate” nucleophiles, particularly designed to effect a carbon-carbon breaking reaction between the two carbonyls of the cross link, as shown in Scheme B below under physiological conditions. This scheme shows the reaction of a prototypic α-dione cleaving agent of the formula I, N-phenacylthiazolium bromide, with an AP-ene-dione derived cross link.

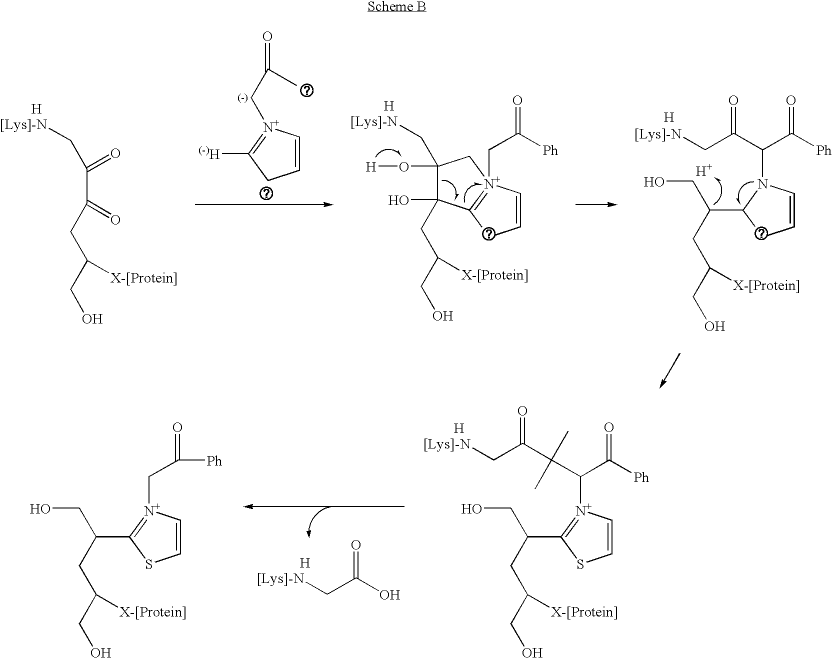

A further experiment to elucidate this reaction involves the reaction of a compound of the formula I, N-phenacyithiazolium bromide, with 1-phenyl-1,2-propanedione to produce the predicted fission product, benzoic acid. The reaction between N-phenacylthiazolium bromide and 1-phenyl-1,2-propanedione was rapid and readily proceeded, confirming this mechanism.

Once early, addition products form on proteins, further reactions can ensue to effect a covalent, protein-protein crosslinking reaction. In this regard, a compound of the formula I, N-phenacylthiazolium bromide, was allowed to react with the AGE-crosslinks that form when AGE-modified BSA (AGE-BSA) is allowed to react with unmodified, native collagen. This resulted in a concentration-dependent release of BSA from the pre-formed AGE-mediated complexes. Again, this confirmed that a significant portion of the AGE-crosslinks that form under experimental conditions consist of an α-diketone or related structure that is susceptible to cleavage by the advantageous bidentate-type molecules of the compounds of formula I under physiological conditions.

The present invention likewise relates to methods for inhibiting the formation of advanced glycosylation endproducts, and reversing the level of already formed advanced glycosylation endproducts, which comprise contacting the target molecules with a composition of the present invention.

The therapeutic implications of the present invention relate to the a method of treating or preventing cardiac disorders associated with a high fat diet or cardiac disorders in patients subjected to a high fat diet.

In the instance where the compositions of the present invention are utilized for in vivo or therapeutic purposes, it may be noted that the compounds used therein are biocompatible. Pharmaceutical compositions may be prepared with a therapeutically effective quantity of the compounds of the present invention and may include a pharmaceutically acceptable carrier, selected from known materials utilized for this purpose. Such compositions may be prepared in a variety of forms, depending on the method of administration. Also, various pharmaceutically acceptable addition salts of the compounds of the invention may be utilized.

A liquid form would be utilized in the instance where administration is by intravenous, intramuscular or intraperitoneal injection. When appropriate, solid dosage forms such as tablets, capsules, or liquid dosage formulations such as solutions and suspensions, etc., may be prepared for oral administration. For topical or dermal application to the skin or eye, a solution, a lotion or ointment may be formulated with the agent in a suitable vehicle such as water, ethanol, propylene glycol, perhaps including a carrier to aid in penetration into the skin or eye. For example, a topical preparation could include up to about 10% of the compound of the invention. Other suitable forms for administration to other body tissues are also contemplated.

In the instance where the present method has therapeutic application, the animal host intended for treatment may have administered to it a quantity of one or more of the compounds, in a suitable pharmaceutical form. Administration may be accomplished by known techniques, such as oral, topical and parenteral techniques such as intradermal, subcutaneous, intravenous or intraperitoneal injection, as well as by other conventional means.

The dose regimen will depend on a number of factors that may readily be determined, such as severity of the condition and responsiveness of the condition to be treated, but will normally be one or more doses per day, with a course of treatment lasting from several days to several months, or until a cure is effected or a diminution of disease state is achieved. One of ordinary skill may readily determine optimum dosages, dosing methodologies, and repetition rates. In general, it is contemplated that the formulation will be administered one to four times daily.

The subject or patient treated by the methods of the invention is a mammal, more preferably a human. The following properties or applications of these methods will essentially be described for humans although they may also be applied to non-human mammals, e.g., apes, monkeys, dogs, mice, etc. The invention therefore can also be used in a plant or veterinarian context.

The compound of the invention is formulated in compositions in an amount effective to inhibit and reverse the formation of advanced glycosylation endproducts. The compound of the invention is formulated in compositions in an amount effective to treat or prevent cardiac disorders associated with a high fat diet or cardiac disorders in patients subjected to a high fat diet. This amount will, of course, vary with the particular agent being utilized and the particular dosage form, but typically is in the range of 0.01% to 1.0%, by weight, of the particular formulation.

The compounds encompassed by the invention are conveniently prepared by chemical syntheses well-known in the art. Certain of the compounds encompassed by the invention are well-known compounds readily available from chemical supply houses and/or are prepared by synthetic methods specifically published therefor. For instance, 3,4-dimethyl-5-(2-hydroxyethyl)thiazolium iodide; 3-ethyl-5-(2-hydroxyethyl)-4-methylthiazolium bromide; 3-benzyl-5-(2-hydroxyethyl)-4-methylthiazolium chloride; and 3-(carboxymethyl)benzothiazolium bromide are obtainable from compounds described in the chemical and patent literature or directly prepared by methods described therein and encompassed by the present invention are those such as 3-(2-phenyl-2-oxoethyl)-4-methylthiazolium bromide and 3-benzyl-5-(2-hydroxyethyl)-4-methyl thiazolium chloride [Potts et al., J. Org. Chem., 41:187-191 (1976)].

Certain of the compounds of the invention are novel compounds, not heretofore known in the art. These compounds are those represented by the formula Ia

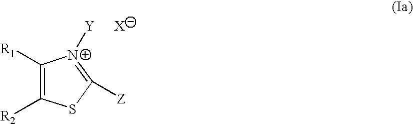

wherein R¹ and R² are independently selected from the group consisting of hydrogen, hydroxy (lower) alkyl, acetoxy(lower)alkyl, lower alkyl, lower alkenyl, or R¹ and R² together with their ring carbons may be an aromatic fused ring, optionally substituted by one or more amino, halo or alkylenedioxy groups;
Z is hydrogen or an amino group;
Y is amino, a group of the formula

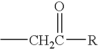

wherein R is a lower alkyl, alkoxy, hydroxy, amino or an aryl group, said aryl group optionally substituted by one or more lower alkyl, lower alkoxy, halo, dialkylamino, hydroxy, nitro or alkylenedioxy groups;
a group of the formula

—CH₂R′

wherein R′ is hydrogen, or a lower alkyl, lower alkynyl, or aryl, group; or a group of the formula

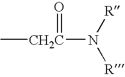

wherein R″ is hydrogen and R″ is a lower alkyl group, optionally substituted by an aryl group, or an aryl group, said aryl group optionally substituted by one or more lower alkyl, halo, or alkoxylcarbonyl groups; or R″ and R′″ are both lower alkyl groups; with the proviso that at least one of Y and Z is an amino group, and the further proviso that when Y is amino and R₂ and Z are both hydrogen, then R₁ is other than a lower alkyl group; and
X is a halide, tosylate, methanesulfonate or methanesulfonate ion.

Other novel compounds are those of formula I wherein Y is a lower alkynylmethyl group or a group of the formula

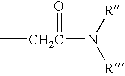

wherein R″ is hydrogen and R″ is a lower alkyl group, optionally substituted by an aryl group, or an aryl group, said aryl group optionally substituted by one or more lower alkyl, halo, or alkoxylcarbonyl groups; or R″ and R″ are both lower alkyl groups.

The compounds of formula I wherein Y is a group of the formula wherein R is a lower alkyl, alkoxy, hydroxy, amino or aryl group;

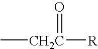

Wherein R is lower alkyl, alkoxy, hydroxy, amino or aryl group; or a group of the formula

—CH₂R′

wherein R′ is hydrogen, or a lower alkyl, lower alkynyl or aryl group;
X is a halide, tosylate, methanesulfonate or mesitylenesulfonate ion; can be prepared according to the methods described in Potts et al., J. Org. Chem., 41:187 (1976); and Potts et al., J. Org. Chem., 42:1648 (1977), or as shown in Scheme I below.

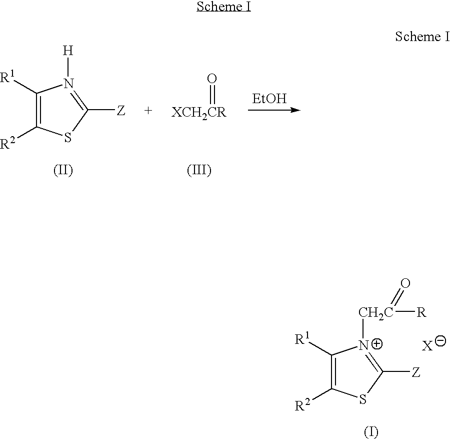

wherein R¹, R², Z, and R are as hereinabove defined, and X is a halogen atom.

In reaction Scheme I, the appropriate substituted thiazole compound of formula II wherein R¹, R² and Z are as hereinbefore defined, is reacted with the appropriate halo compound of. formula III wherein R and X are as hereinbefore defined, to afford the desired compound of the invention e.g., formula I wherein R¹, R², Z, R and X are as hereinbefore defined.

Typically, this reaction is conducted at reflux temperatures for times of about 1-3 hours. Typically, a polar solvent such as ethanol is utilized for the conduct of the reaction.

The compounds of formula I wherein Y is an amino group can be prepared according to the methods described in Tamura et al., Synthesis, 1 (1977), or as shown below in Scheme II.

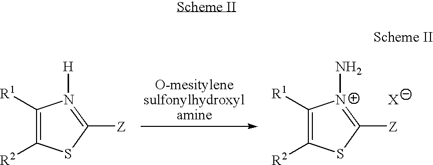

wherein R¹, R² and Z are as defined hereinabove.

In the reaction shown in Scheme II, typically conducted in an anhydrous polar solvent at room temperatures, typical reaction temperatures range from room temperature to reflux, and typical times vary from 1 to about 4 hours. This reaction affords the mesitylene sulfonate, which can then be optionally converted to other thiazolium salts by typical exchange reactions.

The present invention also involves a novel sandwich enzyme immunoassay used to ascertain the ability of test compounds to “break” or reverse already formed advanced glycosylation endproducts by detecting the breaking of AGE (Advanced glycosylation endproduct) moieties from AGE-crosslinked protein. This assay comprises:

a) incubation of AGE-modified bovine serum albumin (AGE BSA) on collagen-coated wells of microtiter plates for a period of 2-6 hours' at a temperature of 37° C.

b) washing of the wells with PBS-Tween;

c) application of the test compounds to the washed wells of step b;

d) incubation of the test compounds applied to the washed wells for an additional 12-24 hours at a temperature of about 37° C.; and

e) detection of the AGE-breaking using an antibody raised against AGE-ribonuclease or cross-link breaking with an antibody against BSA.

Without further elaboration, it is believed that one skilled in the art can, using the preceding description, utilize the present invention to its fullest extent. The following examples are, therefore, to be construed as merely illustrative, and not limitative of the remainder of the disclosure in any way whatsoever.

### EXAMPLE 1

### 3-(2-Methoxy-2-Oxoethyl)-Thiazolium Bromide

Thiazole, (850 mg, 10 mmol), methyl bromoacetate (1.52, 10 mmol) and absolute ethanol (50 ml) were refluxed for 2 hours. On cooling, the salt separated and was recrystallized from absolute ethanol to give the title compound (1.59 g), m.p. 189-190° C. (dec).

### EXAMPLE 2

### 3-Amino-4,5-Dimethylthiazolium Mesitylenesulfonate

An ice cold solution of the 4,5-dimethyl thiazole (2.26 g, 20 mmol) in dry dichloromethane (15 ml) was treated dropwise with a solution of o-mesitylenesulfonylhydroxylamine (4.3 g, 20 mmol) in dry dichloromethane (15 ml). After stirring for 2 hours at room temperature, anhydrous ether (10 ml) was added. On cooling, colorless needles of the title product, 3-amino-4,5-dimethyl-thiazolium mesitylenesulfonate, separated (3.48 g), m.p. 165-168° C.

### EXAMPLE 3

Using the procedures described above in Examples 1 and 2, the following compounds are prepared.
423 3-amino-thiazolium mesitylenesulfonate, m.p. 102-104° C.
427 2,3-diamino-thiazolium mesitylenesulfonate, m.p. 173-175° C. (dec).
670 3-(2-methoxy-2-oxoethyl)-4,5-dimethylthiazolium bromide, m.p. 184-185° C. (dec).
709 3-(2-methoxy-2-oxoethyl)-4-methylthiazolium bromide, m.p. 149-151° C. (dec).
710 3-(2-phenyl-2-oxoethyl)-4-methylthiazolium bromide, m.p. 218-220° C. (dec).
711 3-(2-phenyl-2-oxoethyl)-4,5-dimethylthiazolium bromide, m.p. 212-213° C. (dec).
717 3-amino-4-methyl-thiazolium mesitylene sulfonate, m.p. 143-144° C.
719 3-(2-methoxy-2-oxoethyl)-5-methyl-thiazolium bromide, m.p. 193-194° C. (dec).
720 3-(2-phenyl-2-oxoethyl)-5-methyl-thiazolium bromide, m.p. 193-194° C.
721 3-(2-(4¹-bromophenyl]-2-oxoethyl)-thiazolium bromide, m.p. 269-270° C. (dec).
722 3-(2-[4¹-bromophenyl)-2-oxoethyl)-4-methyl-thiazolium bromide, m.p. 248-249° C. (dec).
723 3-(2-(4¹-bromophenyl]-2-oxoethyl)-5-methyl-thiazolium bromide, m.p. 216-217° C.
724 3-(2-(4-bromophenyl]-2-oxoethyl)-4,5-dimethylthiazolium bromide, m.p. 223-224° C. (dec).
725 3-(2-methoxy-2-oxoethyl)-4-methyl-5-(2-hydroxyethyl)-thiazolium bromide, m.p. 137-138° C.
726 3-(2-phenyl-2-oxoethyl)-4-methyl-5-(2-hydroxyethyl)-thiazolium bromide, m.p. 180-181° C.
727 3-(2-(41-bromophenyl]-2-oxoethyl)-4-methyl-5-(2-hydroxyethyl)thiazolium bromide, m.p. 251-252° C. (dec).
728 3,4-dimethyl-5-(2-hydroxyethyl)-thiazolium iodide, m.p. 85-87° C.
729 3-ethyl-5-(2-hydroxyethyl)-4-methyl thiazolium bromide, m.p. 84-85° C.
730 3-benzyl-5-(2-hydroxyethyl)-4-methyl thiazolium chloride, m.p. 144-146° C.
731 3-(2-methoxy-2-oxoethyl)-benzothiazolium bromide, m.p. 144-145° C. (dec).
732 3-(2-phenyl-2-oxoethyl)-benzothiazolium bromide, m.p.
733 240-241° C. (dec).
734 3-(2-(41-bromophenyl)-2-oxoethyl)-benzothiazolium bromide, m.p. 261-262° C. (dec).
734 3-(carboxymethyl)-benzothiazolium bromide m.p. 250° C. (dec).
735 2,3-diaminio-benzothiazolium mesitylenesulfonate, m.p. 212-214° C. (dec).
738 3-(2-amino-2-oxoethyl)-thiazolium bromide, m.p. 205-206° C.
739 3-(2-amino-2-oxoethyl)-4-methyl-thiazolium bromide, m.p. 220-222° C.
740 3-(2-amino-2-oxoethyl)-5-methyl-thiazolium bromide, m.p. 179-180° C.
741 3-(2-amino-2-oxoethyl)-4,5-dimethyl-thiazolium bromide, m.p. 147-148° C.
742 3-(2-amino-2-oxoethyl)-benzothiazolium bromide, m.p. 222-223° C.
743 3-(2-amino-2-oxoethyl)-4-methyl-5-(2-hydroxyethyl)thiazolium bromide, m.p. 182-183° C.
744 3-amino-5-(2-hydroxyethyl)-4-methyl-thiazolium mesitylenesulfonate, m.p. 94-95° C. (dec).
755 3-(2-methyl-2-oxoethyl)thiazolium chloride, m.p. 178-179° C. 763 3-amino-4-methyl-5-(2-acetoxyethyl)thiazolium mesitylenesulfonate, m.p. 118-120° C.
766 3-(2-phenyl-2-oxoethyl)thiazolium bromide, m.p. 217-218° C.
769 3-(2-methoxy-2-oxoethyl)-4-methyl-5-(2-acetoxyethyl)thiazolium bromide, m.p. 217-218° C.
770 3-(2-amino-2-oxoethyl)-4-methyl-5-(2-acetoxyethyl)thiazolium bromide, m.p. 233-234° C.
771 2-amino-3-(2-methoxy-2-oxoethyl)thiazolium bromide, m.p. 191-192° C.
772 2-amino-3-(2-methoxy-2-oxoethyl)benzothiazolium bromide, m.p. 236-237° C.
773 2-amino-3-(2-amino-2-oxoethyl)thiazolium bromide, m.p. 209-210° C.
774 2-amino-3-(2-amino-2-oxoethyl)benzothiazolium bromide, m.p. 234-235° C.
798 3-(2-(4′-methoxyphenyl)-2-oxoethy].]-thiazolinium bromide, m.p. 248-249° C. (dec.);
799 3-(2-(2′,4′-dimethoxyphenyl)-2-oxoethyl]-thiazolinium bromide, m.p. 214-216° C. (dec.); 35 800 3-(2-(4′-fluorophenyl-2-oxoethyl]-thiazolinium bromide, m.p. 209-210° C. (dec.);
801 3-(2-(2′,4′-difluorophenyl)-2-oxoeethyl)-thiazolinium bromide, m.p. 226-228° C. (dec.);
802 3-(2-(4′-diethylaminophenyl)-2-oxoethyl]-thiazolinium bromide, m.p. 233-235° C. (dec.);
803 3-propargyl-thiazolium bromide, m.p. 64-66° C.;
804 3-Propargyl-4-methyl thiazolium bromide, m.p. 213-215° C.;
805 3-Propargyl-5-methyl thiazolium bromide, m.p. 127-129° C.;
806 3-Propargyl-4,5-dimethyl thiazolium bromide, m.p. 198-200° C.;
807 3-Propargyl-4-methyl-5-(2-hydroxyethyl)-thiazolium bromide, m.p. 132-134° C.;
824 3-(2-(3′-methoxyphenyl]-2-oxoethyl)-thiazolium bromide, m.p. 224-225° C.;
825 3-(2-[3′-methoxyphenyl]-2-oxoethyl)-4 methyl 5-(2′-hydroxyethyl)-thiazolium bromide. m.p. 164-165° C.;
826 3-(2-[3′-methoxyphenyl]-2-oxoethyl)-benzothiazolium bromide, m.p. 215-217° C.;
836 2,3-diamino-4-chlorobenzothiazolium mesitylenesulfonate, m.p. 228-230° C.;
847 2,3-diamino-4-methyl-thiazolium mesitylene sulfonate, m.p. 204-205° C.;
848 3-amino-4-methyl-5-vinyl-thiazolium mesitylene sulfonate, m.p. 145-147° C.;
858 2,3-diamino-6-chlorobenzothiazolium mesitylenesulfonate, m.p. 244-246° C.;
862 2,6-diamino-benzothiazole dihydrochloride, m.p. 318-320° C. (dec.);
876 2,6-diamino-3 (2-(4′-methoxyphenyl)-2-oxoethyl]benzothiazolium bromide, m.p. 243-245° C. (dec.);
877 2,6-diamino-3 (2-(3′-methoxyphenyl)-2-′oxoethyl]benzothiazolium bromide, m.p. 217-218° C. (dec.);
878 2,6-diamino-3 (2-(4′-diethylaminophenyl)-2-oxoethyl)benzothiazolium bromide, m.p. 223-225° C. (dec.);
887 2,6-diamino-3 (2-(4′-bromophenyl)-2-oxoethyl]benzothiazolium bromide, m.p. 258-259° C. (dec.);
888 2,6-diamino-3 (2-(2-phenyl-2-oxoethyl)benzothiazolium bromide, m.p. 208-210° C. (dec.);
889 2,6-diamino-3 (2-(4′-fluorophenyl-2-oxoethyl]benzothiazolium bromide, m.p. 251-252° C. (dec.);
897 3-acetamido-4-methyl-5-thiazolyl-ethyl acetate mesitylenesulfonate, m.p. syrup material;
913 2,3-diamino-5-methylthiazolium mesitylenesulfonate, m.p. 149-152° C.;
924 3-(2-(2′-naphthyl)-2-oxoethyl]-4-methyl-5-(2′-hydroxyethyl)-thiazolium bromide, m.p. 219-220° C.;
925 3-(2-(3′,5′-Di-tert-butyl-4′-hydroxyphenyl)-2-15 oxoethyl]-4-methyl-5-(2′-hydroxyethyl)-thiazolium bromide, m.p. 206-207° C.;
928 3-[2-(2′,6′-Dichlorophenethylamino)-2-oxoethyl]-4-methyl-5-(2′-hydroxyethyl)-thiazolium bromide, m.p. 193-195° C.;
929 3-(2-Dibutylamino-2-oxoethyl]-4-methyl-5-(2′-hydroxyethyl)-thiazolium bromide, m.p. 78-80° C.;
930 3-(2-4′-carbethoxyanilino)-2-oxoethyl]-4-methyl-5-(2′-hydroxyethyl)-thiazolium bromide, m.p. 204-206° C.;
931 3-(2-(2′,6′-Diisopropylanilino)-2-oxoethyl]-4-methyl-5-(2′-hydroxyethyl)-thiazolium bromide, m.p. 166-168° C.;
932 3-amino-4-methyl-5-(2(2′,6′-dichlorobenzyloxy)ethyl]-thiazolium mesitylenesulfonate, 30 m.p. 164-166° C.;
935 3-(2-(4′-carbmethoxy-3′-hydroxyanilino)-2-oxoethyl]-4-methyl-5-(2′-hydroxyethyl)-thiazolium bromide, m.p. 222-223° C.;
938 2,3-Diamino-4,5-dimethyl thiazolium mesitylene sulfonate, m.p. 166-168° C.;
939 2,3-Diamino-4-methyl-5-hydroxyethyl-thiazolium mesitylene sulfonate, m.p. 132-134° C.;
940 2,3-Diamino-5-(3′,4′-trimethylenedioxy phenyl)thiazolium mesitylene sulfonate, m.p. 224-226° C.;
941 3 (2-(1′,4′-benzodioxan-6-yl)-2-oxoethyl]-4-methyl-5-(2′-hydroxyethyl)-thiazolium bromide, m.p. 196-198° C.;
942 3-(2-(3′,4′-trimethylenedioxyphenyl)-2-oxoethyl]-4-methyl-5-(2′-hydroxyethyl)-thiazolium bromide, m.p. 164-166° C.;
943 3-(2-(1′,4-benzodioxan-6-yl]-2-oxoethyl)-thiazolium bromide, m.p. 238-239° C.;
944 3-(2-(3′,4′-trimethylenedioxyphenyl)-2-oxoethyl]-thiazolium bromide, m.p. 246-248° C. (dec.);
948 3-[2-(3′,5″-di-tert-butyl-4′-hydroxyphenyl)-2-oxoethyl]-thiazolium bromide, m.p.
949 3-[2-(3′,5′-di-tert-butyl-4′-hydroxyphenyl)-2-oxoethyl]-4-methyl-thiazolium bromide, m.p. 226-228° C. (dec.);
950 3-[2-(3′,5′-di-tert-butyl-4′-hydroxyphenyl)-2-oxoethyl)-5-methyl-thiazolium bromide, m.p. 210-211° C.;
951 3-(2-(3′,5′-di-tert-butyl-4′-hydroxypheny].)-2-oxoethyl]-4,5-dimethyl-thiazolium bromide, m.p. 243-244° C. (dec.);
952 3-(2-(3′,5′-di-tert-butyl-4′-hydroxyphenyl)-2-oxoethyl]-benzothiazolium bromide, m.p. 239-294° C. (dec.);
953 1-methyl-3-(2-(3′,5′-di-tert-butyl-4′-hydroxyphenyl)-2-oxoethyl]-imidazolium bromide, m.p. 148-150° C.;
954 3-[2-(4′-n-pentylphenyl)-2-oxoethyl]-thiazolinium bromide, m.p. 218-220° C. (dec.);
955 3-(2-(4′-n-pentylphenyl)-2-oxoethyl]-4-methyl-5-(2′-hydroxyethyl)-thiazolinium, m.p. 178-180° C. (dec.);
956 3-(2-4′-diethylaminophenyl)-2-oxoethyl]-4-qj. methyl-5-(2′-hydroxyethyl)-thiazolinium bromide, m.p. 184-186° C. (dec.);
957 3-(2-phenyl-2-oxoethyl)-4-methyl-5-vinyl-thiazolium bromide, m.p. 176-177° C.;
958 3-(2-(3′,5′-tert-butyl-4′-hydroxyphenyl)-2-oxoethyl)-4-methyl-5-vinyl-thiazolium bromide, m.p. 208-209° C.;
959 3-(2-tert-butyl-2-oxoethyl)-thiazolium bromide, m.p. 211-212° C.;
960 3-(2-tert-butyl-2-oxoethyl)-4-methyl-5-(2′-hydroxyethyl)-thiazolium bromide, m.p. 186-187° C.;
961 3-(3′-methoxybenzyl)-4-methyl-5-(2′-hydroxyethyl)-thiazolium chloride, m.p. 135-136° C.;
962 3-(2′,6′-dichlorobenzyl)-4-methyl-5-(2′-15 hydroxyethyl)-thiazolium chloride, m.p. 192-194° C.;
963 3-(2′-nitrobenzyl)-4-methyl-5-(2′-hydroxyethyl)-thiazolium bromide, m.p. 215-216° C.;
964 3 (2-(4′-chlorophenyl)-2-oxoethyl]-thiazolium bromide, m.p. 239-241° C. (dec.);
965 3 (2-(4′-chlorophenyl)-2-oxoethyl]-4-methyl-5-(2′-hydroxyethyl)-thiazolium bromide, m.p. 240-251° C. (dec.); and
966 3 (2-(4′-methoxyphenyl)-2-oxoethyl]-4-methyl-5-(2′ hydroxyethyl)-thiazolium bromide, m.p. 229-231° C. 25 (dec.).

### EXAMPLE 4

| | Mg/tablet |
|---|---|
| Compound of Formula I | 50 |
| Starch | 50 |
| Mannitol | 75 |
| Magnesium stearate | 2 |
| Stearic acid | 5 |

The compound, a portion of the starch and the lactose are combined and wet granulated with starch paste. The wet granulation is placed on trays and allowed to dry overnight at a temperature of 45° C. The dried granulation is comminuted in a comminutor to a particle size of approximately 20 mesh. Magnesium stearate, stearic acid and the balance of the starch are added and the entire mix blended prior to compression on a suitable tablet press. The tablets are compressed at a weight of 232 mg. using a 11/32″ punch with a hardness of 4 kg. These tablets will disintegrate within a half hour according to the method described in USP XVI.

### EXAMPLE 5

| Lotion | mg/g |
|---|---|
| Compound of Formula I | 1.0 |
| Ethyl alcohol | 400.0 |
| Polyethylene glycol 400 | 300.0 |
| Hydroxypropyl cellulose | 5.0 |
| Propylene glycol | to make 1.0 g |

### EXAMPLE 6

Oral Rinse

| | |
|---|---|
| Compound of Formula I: | 1.4% |
| Chlorhexidine gluconate | 0.12% |
| Ethanol | 11.6% |
| Sodium saccharin | 0.15% |
| FD&C Blue No. 1 | 0.001% |
| Peppermint Oil | 0.5% |
| Glycerine | 10.0% |
| Tween 60 | 0.3% |
| Water to | 100% |

### EXAMPLE 7

Toothpaste

| | |
|---|---|
| Compound of Formula I: | 5.5% |
| Sorbitol, 70% in water | 25% |
| Sodium saccharin | 0.15% |
| Sodium lauryl sulfate | 1.75% |
| Carbopol 934, 6% dispersion in | 15% |
| Oil of Spearmint | 1.0% |
| Sodium hydroxide, 50% in water | 0.76% |
| Dibasic calcium phosphate dihydrate | 45% |
| Water to | 100% |

### EXAMPLE 8

### Cross-Linking Inhibition Assay

The following method was used to evaluate the ability of the compounds of the present invention to inhibit the cross-linking of glycated bovine serum albumin (AGE-BSA) to the rat tail tendon collagen-coated 96-well plate.

The AGE-BSA was prepared by incubating BSA at a concentration of 200 mg per ml with 200 mM glucose in 0.4M sodium phosphate buffer, pH 7.4 at 37° C. for 12 weeks. The glycated ESA was then extensively dialyzed against phosphate buffer solution (PBS) for 48 hours with additional 5 times buffer exchanges. The rat tail tendon collagen coated plate was blocked first with 300 ml of superbloc blocking buffer (Pierce #37515×) for one hour. The blocking solution was removed from the wells by, washing the plate twice with PBS-'Tween 20 solution (0.05% Tween 20) using a NUNq-multiprobe or Dynatech ELISA-plate washer. Cross-linking of AGE-BSA (1 to 10 mg per well depending on the batch of AGE-BSA) to rat tail tendon collagen coated plate was performed with and without the testing ‘compound dissolved in PBS buffer at pH 7.4 at the desired concentrations by the, addition of 50 μl each of the AGE-BSA diluted in PBS or in the solution of test compound at 37° C. for 4 hours.

Unbrowned BSA in PBS buffer with or without testing compound were added to the separate wells as the blanks. The un-cross-linked AGE-BSA was then removed by washing the wells three times with PBS-Tween buffer. The amount of AGE-BSA cross-linked to the tail tendon collagen-coated plate was then quantitated using a polyclonal antibody raised against AGE-RNase. After a one-hour incubation period, AGE antibody was removed by washing 4 times with PBS-Tween.

The bound AGE antibody was then detected with the addition of horseradish peroxidase-conjugated secondary antibody—goat anti-rabbit immunoglobulin and incubation for 30 minutes. The substrate of 2,2-azino-di(3-ethylbenzthiazoline sulfonic acid) (ABTS chromogen) (Zymed #00-2011) was added. The reaction was allowed for an additional 15 minutes and the absorbance was read at 410 nm in a Dynatech plate reader.

The % inhibition of each test compound was calculated as 15 follows. % inhibition={(Optical density (without compound)−optical density (with compound)]/optical density (without compound))×100%

The IC₅₀ values or the inhibition at various concentrations by test compounds is as follows:

| | | Relative Cross-link |
|---|---|---|
| Test Compound | IC₅₀ | Inhibition |
| Inhibition Data | (mM) | (at 10 mM) |
| 3-amino-4,5-dimethylaminothiazolium mesitylenesulfonate | 2.8 | |
| 2,3-diaminothiazolinium mesitylenesulfonate | >.10 | 27% |
| 3-(2-methoxy-2-oxoethyl)-thiazolium bromide | 0.25 | |
| 3-(2-methoxy-2-oxoethyl)-4,5-dimethylthiazolium bromide | 0.48 | |
| 3-(2-methoxy-2-oxoethyl)-4-methylthiazolium bromide | | 58% |
| 3-(2-phenyl-2-oxoethyl)-4-methylthiazolium bromide | 5.6 | |
| 3-(2-phenyl-2-oxoethyl)-4,5-dimethylthiazolium bromide | | 37% |
| 3-amino-4-methylthiazolium mesitylenesulfonate | | 46% |
| 3-(2-methoxy-2-oxoethyl)-5-methylthiazolium bromide | 3.2 | |
| 3-(3-(2-phenyl-2-oxoethyl)-5-methylthiazolium bromide | 12.6 | |
| 3-[2-(4′-bromophenyl)-2-oxoethyl]-4-methylthiazolium | | 37% |
| bromide | | |
| 3-[2-(4′ bromophenyl)-2-oxoethyl]-4,5- | 2.92 | |
| dimethylthiazolium | | |
| 3-(2-methoxy-2-oxoethyl)-4-methyl-5-(2-hydroxyethyl) | | 38% |
| thiazolium bromide | | |
| 3-(2-phenyl-2-oxoethyl)-4-methyl-5-(2-hydroxyethyl) | >10 | 36% |
| thiazolium bromide | | |
| 3-[2-(4′-bromophenyl)-2-oxoethyl]-4-methyl-5-(2- | 2.95 | |
| hydroxyethyl) thiazolium bromide | | |
| 3-(2-methoxy-2-oxoethyl) benzothiazolium bromide | >10 | 35% |
| 3-(carboxymethyl) benzothiazolium bromide | | 16% |
| 2,3-(diamino) benzothiazolium mesitylenesulfonate | 0.0749 | |
| 3-(2-amino-2-oxoethyl) thiazolium bromide | 0.53 | |
| 3-(2-amino-2-oxoethyl)-4-methylthiazolium bromide | 0.7 | |
| 3-(2-amino-2-oxoethyl)-5-methylthiazolium bromide | 0.0289 | |
| 3-(2-amino-2-oxoethyl) 4,5-dimethylthiazolium bromide | 9.9 | |
| 3-(2-amino-2-oxoethyl) benzothiazolium bromide | 0.02 | |
| 3-(2-amino-2-oxoethyl) 4-methyl-5-(2-hydroxyethyl) | 1.42 | |
| thiazolium bromide | | |
| 3-amino-5-(2-hydroxyethyl)-4-methylthiazolium | 3.6 × 10⁵ | |
| mesitylenesulfonate | | |
| 3-(2-phenyl-2-oxoethyl) thiazolium bromide | 11.1 | 34% |
| 3-(2-[3′-methoxyphenyl]-2-oxoethyl-thiazolium bromide | | 29% |
| 2,3-diamino-4-chlorobenzothiazolium mesitylenesulfonate | | 33% |
| 2,3-diamino-4-methyl-thiazolium mesitylenesulfonate | | 40% |
| 3-amino-4-methyl-5-vinyl-thiazolium mesitylenesulfonate | 11.3 | |
| 2,3-diamino-6-chlorobenzothiazolium mesitylenesulfonate | | 23.2 (2 mm) |
| 2,6-diamino-3[2-(4′-methoxyphenyl)-2-oxoethyl] | | |
| benzothiazolium bromide | | |
| 2,6-diamino-3[2-(4′-bromophenyl)-2-oxoethyl] | | |
| benzothiazolium bromide | | |
| 2,6-diamino-3[2-(4′-fluorophenyl-2-oxoethyl] | | |
| benzothiazolium bromide | | |
| 2,3-diamino-5-methylthiazolium mesitylenesulfonate | | |
| 3-[2-(2′-naphthyl)-2-oxoethyl]-4-methyl-5-(2′-hydroxy- | | 61% |
| ethyl)-thiazolium bromide | | |
| 3-[2-Dibutylamino-2-oxoethyl]-4-methyl-5-(2′- | | 0.8% (10 mm) |
| hydroxyethyl)-thiazolium bromide | | |
| 3-[2-4′-carbethoxyanilino)-2-oxoethyl]-4-methyl-5-(2′- | | 8.8% (1 mm) |
| hydroxyethyl)-thiazolium bromide | | |
| 3-[2-(2′,6′-Diisopropylanilino)-2-oxoethyl]-4-methyl-5- | | 19% |
| (2′-hydroxyethyl)-thiazolium bromide | | |
| 3-amino-4-methyl-5-[2(2′,6′-dichlorobenzyloxy) ethyl]- | | 26.5% (3 mm) |
| thiazolium mesitylenesulfonate | | |
| 3-[2-(4′-carbmethoxy-3′-hydroxyanilino)-2-oxoethyl]- | 1.76 | |
| 4-methyl-5-(2′-hydroxyethyl)-thiazolium bromide | | |
| 2,3-Diamino-4,5-dimdethyl thiazolium mesitylene | | 39% |
| sulfonate | | |
| 2,3-Diamino-4-methyl-5-hydroxyethyl-thiazolium | | 18% |
| mesitylene sulfonate | | |
| 2,3-Diamino-5-(3′,4′-trimethylenedioxy phenyl)- | | 40% @ 3 mM |
| thiazolium mesitylene sulfonate | | |
| 3[2-(1′,4′-benzodioxan-6-yl)-2-oxoethyl]-4-methyl-5- | | 13% |
| (2′-hydroxyethyl)-thiazolium bromide | | |
| 3-[2-(3′,4′-trimethylenedioxyphenyl)-2-oxoethyl]- | | 4.4 |
| thiazolium bromide | | |
| 3-[2-(3′,4′-trimethylenedioxyphenyl)-2-oxoethyl]- | | 45% |
| thiazolium bromide | | |
| 3-[2-(3′,5′-di-tert-butyl-4′-hydroxyphenyl)-2-oxoethyl]- | | 24% @ 0.3 mM |
| 4-methyl-thiazolium bromide | | |
| 3-[2-(3′,5′-di-tert-butyl-4′-hydroxyphenyl)-2-oxoethyl]- | 0.78 | 69% @ 1 mM |
| 5-methyl-thiazolium bromide | | |
| 3-[2-(3′,5′-di-tert-butyl-4′-hydroxyphenyl)-2-oxoethyl]- | 0.16 | |
| 4,5-dimethyl-thiazolium bromide | | |
| 1-methyl-3-[2-(3′,5′-di-tert-butyl-4′-hydroxyphenyl)-2- | 4.5 | |
| oxoethyl]-imidazolium bromide | | |
| 3-[2-(4′-n-pentylphenyl)-2-oxoethyl]-thiazolinium | | ND |
| bromide | | |
| 3-[2-(4′-n-pentylphenyl)-2-oxoethyl]-4-methyl-5-(2′- | 1.53 | 52% @ 3 mM |
| hydroxyethyl)-thiazolinium bromide | | |
| 3-[2-4′-diethylaminophenyl)-2-oxoethyl]-4-methyl-5- | 2.8 | |
| (2′-hydroxyethyl)-thiazolinium bromide | | |
| 3-(2-phenyl-2-oxoethyl)-4-methyl-5-vinyl-thiazolium | ND | |
| bromide | | |
| 3-[2-(3′,5′-tert-butyl-4′-hydroxyphenyl)-2-oxoethyl)-4- | ND | |
| methyl-5-vinyl-thiazolium bromide | | |

The above experiments suggest that this type of drug therapy may have benefit in reducing the pathology associated with the advanced glycosylation of proteins and the formation of cross-links between proteins and other macromolecules. Drug therapy may be used to treat or prevent endothelial dysfunction or NO-dependant vasodilation prevent Both topical, oral, and parenteral routes of administration to provide therapy locally and systemically are contemplated.

### EXAMPLE 9

### Cross-Link Breaking Assay

In order to ascertain the ability of the compounds of the instant invention to “break” or reverse already formed advanced glycosylation endproducts, a novel sandwich enzyme immunoassay was developed which detects breaking of AGE (Advanced glycosylation endproduct) moieties from AGE-crosslinked protein. The assay utilizes collagen-coated 96 well microtiter plates that are obtained commercially. AGE-modified protein (AGE-BSA), prepared, for instance, as in Example 8, above, is incubated on the collagen-coated wells for four hours, is washed off the wells with PBS-Tween and solutions of the test compounds are added. Following an incubation period of 16 hours (37° C.) cross-link-breaking is detected using an antibody raised against AGE-ribonuclease or with an antibody against BSA. Positive results in this assay indicate compounds that are capable of reducing the amount of AGE-BSA previously crosslinked to the collagen by breaking the crosslinks and allowing the liberated material to be flushed away in subsequent washing steps. Details of the assay are as follows:

### Materials

### Immunochemicals and Chemicals

### Bovine Serum Albumin (Type V), (BSA) Calbiochem

### Dextrose

### Superbloc, Pierce, Inc.

Rabbit anti-Bovine Serum Albumin

### Horseradish Peroxidase (HRP)-Goat-anti-rabbit), Zymed

HRP substrate buffer, Zymed
ABTS chromogen, Zymed

### Phosphate Buffer Saline

### Tween 20, Sigma

### Equipment

### ELISA Plate Washer, Dynatech

### ELISA Plate Reader, Dynatech

### Precision Water Bath

Corning digital pH meter

### Glassware and Plasticware

### Finneppette Multichannel Pipettor, Baxter

Eppendorf pipettes, Baxter
Eppendorf repeater pipette, Baxter
Pipettor tips for Finneppetter, Baxter
Pipettor tips for Eppendorf, Baxter
Glass test tubes, 13×100 mm; Baxter
Mylar Sealing Tape for 96 well plates, Corning
Biocoat Cellware Rat Tail Collagen Type-1 coated 96-well plates, Collaborative Biomedical Products.

### Methods

### Preparation of Solutions and Buffers

1. AGE-BSA stock solutions were prepared as follows. Sodium phosphate buffer (0.4 M) was prepared by dissolving 6 grams of monobasic sodium phosphate in 100 ml of distilled water, 7 grams of dibasic sodium phosphate (0.4 M) in 100 ml of distilled water and adjusting the pH of the dibasic solution to 7.4 with the monobasic solution. Sodium azide (0.02 grams) was added per 100 ml volume to inhibit bacterial growth. The BSA solution was prepared as follows: 400 mg of Type V BSA (bovine serum albumin) was added for each ml of sodium phosphate buffer (above). A 400 mM glucose solution was prepared by dissolving 7.2 grams of dextrose in 100 ml of sodium phosphate buffer (above). The BSA and glucose solutions were mixed 1:1 and incubated at 37° C. for 12 weeks. The pH of the incubation mixture was monitored weekly and adjusted to pH 7.4 if necessary. After 12 weeks, the AGE-BSA solution was dialyzed against PBS for 48 hours with four buffer changes, each at a 1:500 ratio of solution to dialysis buffer. Protein concentration was determined by the micro-Lowry method. The AGE-BSA stock solution was aliquoted and stored at −20° C. Dilute solutions of AGE-BSA were unstable when stored at −20° C.

2. Working solutions for crosslinking and breaking studies were prepared as follows. Test compounds were dissolved in PBS and the pH was adjusted to pH 7.4 if necessary. AGE-BSA stock solution was diluted in PBS to measure maximum crosslinking and in the inhibitor solution for testing inhibitory activity of compounds. The concentration of AGE-BSA necessary to achieve the optimum sensitivity was determined by initial titration of each lot of AGE-BSA.

3. Wash buffer (“PBS-Tween”) was, prepared as follows. PBS was prepared by dissolving the following salts in one liter of distilled water: NaCl, 8 grams; KCl, 0.2 gram, KH₂PO₄. 1.15 grams; NaN₃, 0.2 gram. Tween-20 was added to a final concentration of 0.05% (vol/vol).

4. Substrates for detection of secondary antibody binding were prepared by diluting the HRP substrate buffer 1:10 in distilled water and mixing with ABTS chromogen 1:50 just prior to use.

### Assay Procedures

1. Biocoat plates were blocked with 300 μl of “Superbloc”. Plates were blocked for one hour at room temperature and were washed with PBS-Tween three times with the Dynatech platewasher before addition of test reagents.

2. Each experiment was set up in the following manner. The first three wells of the Biocoat plate were used for the reagent blank. Fifty microliters of solutions AGE-BSA were added to test wells in triplicate and only PBS in blank wells. The plate was incubated at 37° C. for four hours and washed with PBS-Tween three times. Fifty microliters of PBS was added to the control wells and 50 μl of the test “AGE Cross-link breaker” compound was added to the test wells and blank. The plate was incubated overnight (approximately 16 hours) with the test “AGE Cross-link breaker” compound, followed by washing in PBS before addition of primary antibody (below).

3. Each lot of primary antibody, either anti-BSA or anti-RNase, was tested for optimum binding capacity in this assay by preparing serial dilutions (1:500 to 1:2000) and plating 50 μl of each dilution in the wells of Biocoat plates. Optimum primary antibody was determined from saturation kinetics. Fifty microliters of primary antibody of appropriate dilution, determined by initial titration, was added and incubated for one hour at room temperature. The plate was then washed with PBS-Tween.

4. Plates were incubated with the secondary antibody, HRP (Goat-anti-rabbit), which was diluted 1:4000 in PBS and used as the final secondary antibody. The incubation was performed at room temperature for thirty minutes.

5. Detection of maximum crosslinking and breaking of AGE crosslinking was performed as follows. HRP substrate (100 ul) was added to each well of the plate and was incubated at 37° C. for fifteen minutes. Readings were taken in the Dynatech ELISA-plate reader. The sample filter was set to “1” and the reference filter ‘was set to “5”.

### Standard Operating Procedure

### Preliminary Steps

1. Titrate each new lot of AGE-BSA preparation as described in Table 4 and determine the optimum AGE-BSA concentration for the ELISA assay from saturation kinetics.

2. At the beginning of the day, flush the plate washer head with hot water, rinse with distilled water and 50% ethanol. Fill the buffer reservoir of the plate washer with PBS-Tween (0.05%) and purge the system three times before use.

3. Prepare an assay template for setting up the experiment as described under “Assay Setup”, #2, below.

### Assay Setup

1. Warm Superbloc reagent to 37° C. Add 300 μl of Superbloc to each well of the Biocoat plate and let stand for sixty minutes at 37° C. Wash the wells three times with PBS-Tween (0.05%). Turn the plate 180 degrees and repeat this wash cycle.

2. Dilute the AGE-BSA in PBS so that 50 μl of the diluted sample will contain the amount of AGE-BSA necessary for minimum crosslinking and inhibition by pimagedine (aminoguanidine), as determined by initial titration described above. Prepare negative controls by dissolving non-browned BSA in PBS at the same concentration as the AGE-BSA. Add 50 μl of AGE-BSA or BSA to each well which correspond to the “AGE-BSA” and “BSA” labels on the template.

3. Dissolve the test compounds in PBS at 30 mM concentration for preliminary evaluation. The pH must be checked and adjusted to 7.4 when necessary. Pretreat the collagen-coated plates with AGE-BSA to obtain maximum crosslinking. Prepare negative controls for inhibition experiments by dissolving BSA in the inhibition solution at the same protein concentration as that used for AGE-BSA. Add 50 μl of AGE-BSA or BSA in the inhibitor solutions to the wells which correspond to “test compound+AGE-BSA and to “test compound blank”, respectively, on the template. Incubate the plate at 37° C. for four hours. Following covalent binding of AGE-BSA to the plates, wash the plates with PBS-Tween in preparation of the detection reaction (below).

4. Binding of primary antibody to the Biocoat plates is carried out as follows. At the end of the four hour incubation, the wells are washed with PBS-Tween. Appropriate dilutions (as determined by initial titration) of the rabbit-anti-AGE-RNase or rabbit-anti-BSA antibodies were prepared in PBS, and 50 μl is added to each well and the plate is allowed to stand at room temperature for sixty minutes.

5. Secondary antibody binding wells are washed with PBS— Tween and 50 microliters HRP (Horseradish Periodase) (Goat anti-rabbit serum) diluted to 1-4000 in PBS and is added to each well. The plate is allowed to stand at room temperature for 30 minutes.

6. Color development was carried out as follows. Plates are washed as in Step 4 above. Dilute the HRP-substrate buffer 1:10 in water. Add 200 μl of ABTS solution, mix well and add 100 μl of this reagent to each well. Incubate the plate at 37° C. for 15 minutes. Read the optical density at 410 run with the sample filter set to “1” and the reference filter set to “5” on the Dynatech ELISA plate reader. Calculate the percent inhibition by the compound as described above. Compounds which are found to reduce the amount of immunoreactivity are considered to be therapeutically useful insofar as they reverse and reduce the levels of advanced glycosylation endproducts.

| | IC₅₀ (mM) | |
|---|---|---|
| | Anti- | Breaking Anti- |
| | AGE/Anti- | AGE/Anti-BSA (at |
| Test Compound | BSA | mM) |
| 3-aminothiazolium mesitylenesulfonate | 0.005/3.0 | 71%/67% (30) |
| 3-amino-4,5dimenthylaminothiazolium | | 63%/44% (10) |
| mesitylenesulfonate | | |
| 2,3-diminothiazolinium mesitylenesulfonate | 0.28/0.18 | 79%/90% (10) |
| 3-(2-methoxy-2-oxoethyl)-thiazolium bromide | | 38%/41% (30) |
| 3-(2-methoxy-2-oxoethyl)-4,5-dimethylthiazolium | | 63%/47% (30) |
| bromide | | |
| 3-(2-methoxy-2-oxoethyl)-4-methylthiazolium bromide | | 54%/51% (30) |
| 3-(2-phenyl-2-oxoethyl)-4-methylthiazoliumbromide | 0.23/0.30 | 68%/66% (30) |
| 3-(2-phenyl-2-oxoethyl)-4,5-dimethylthiazolium | | 56%/ND (30) |
| bromide | | |
| 3-amino-4-methylthiazolium mesithylenesulfonate | | 55%/ND (30) |
| 3-(2-methoxy-2-oxoethyl)-5-methylthiazolium bromide | | 72%/27% (30) |
| 3-[2-(4′-bromophenyl)-2-oxoethyl) thiazolium bromide | | 76%/25% (30) |
| 3-(2-phenyl-2-oxoethyl)-4-methyl-5-(2- | 14.3/112.0 | 67%/13% (30) |
| hyroxyethyl)thiazolium bromide | | |
| 3-benzyl-5-(2-hydroxyethyl)-4-methylthiazolium | 0.42/0.55 | 65%/61% (30) |
| chloride | | |
| 3-(2-methoxy-2-oxoethyl)benzothiazolium bromide | 1.20/25.9 | 66%/37% (30) |
| 3-(carboxymethyl) benzothiazolium bromide | | 63.7%/17.9% (30) |
| 2,3-(diamino) benzothiazolium mesithylenesulfonate | | 87%/54% (30) |
| 3-(2-amino-2-oxoethyl)-4-methylthiazolium bromide | 4.70/38.6 | 89%/44% (30) |
| 3-(2-amino-2-oxoethyl)4,5-dimethylthiazolium | | 61%/16% (30) |
| bromide | | |
| 3-(2-amino-2-oxoethyl) benzothiazolium bromide | 0.4/0.52 | 77%/65% |
| 3-(2-amino-2-oxoethyl) 4-methyl-5-(2- | 0.012/0.120 | 65%/57% |
| hydroxyethyl)thiazolium bromide | | |
| 3-amino-5-(2-hydroxyethyl)-4-methylthiazoium | 0.18/0.50 | 76%/48% |
| mesitylenesulfonate | | |
| 3-(2-methyl-2-oxoethyl) thiazolium chloride | 0.83/0.75 | 56%/93% |
| 3-(2-phenyl-2-oxoethyl) thiazolium bromide | 0.020/0.014 | 73%/98% |
| 3-(2-[3′-methoxyphenyl]-2-oxoethyl)-thiazolium | | 22%/44% (10) |
| bromide | | |
| 2,3-diamino-4-chorobenzothiazolium | | 21%/26 (10) |
| mesitylenesulfonate | | |
| 2,3-diamino-4-methyl-thiazolium mesithylenesulfonate | | 25%/30% (10) |
| 3-amino-4-methyl-5-vinyl-thiazolium | ND/2.0 | 51%/74% (10) |
| mesitylenesulfonate | | |
| 2,3-diamino-6-chlorobenzothiazolium | | 25%/51 (10) |
| mesithylenesulfonate | | |
| 2,6-diamino-3[2-(4′-methoxyphenyl)-2-oxoethyl] | | 29%/35% (10) |
| benzothiazolium bromide | | |
| 2,6-diamino-3 (2-(4′-bromophenyl)-2-oxoethyl)] | | 27%/44% (10) |
| benzothiazolium bromide | | |
| 2,6-diamino-3 [2-(4′-fluorophenyl-2-oxoethyl] | | 24%/40% (10) |
| benzothiazolium bromide | | |
| 2,3-diamino-5-methylthiazolium mesitylenesulfonate | | 14%/17% (10) |
| 3-[2-(2′-naphthyl)-2-oxoethyl]-4-methyl-5-(2′- | | 52%/61% (10) |
| hydroxyethyl)-thiazolium bromide | | |
| 3-[Dibutylamino-2-oxoethyl]-4-methyl-5-(1′- | | 25%/38% (10) |
| hydroxyethyl)-thiazolium bromide | | |
| 3-[2-4′-carbethoxyanilino)-2-oxoethyl]-4-methyl-5-(2′- | | 48%-57% (10) |
| hydroxtethyl)-thiazolium bromide | | |
| 3-[2-(2′,6′-Diisopropylanilino)-2-oxoethyl]-4-methyl- | | 31%/48% (10) |
| 5-(2′-dhyroxyethyl)-thiazolium bromide | | |
| 3-amino-4-methyl-5-[2(2′,6′-dichlorobenzyloxy)ethyl]- | | 31%/54% (10) |
| thiazolium mesitylenesulfonate | | |
| 3-[2-(4′-carbmethoxy-3′-hydroxyanilino)-2-oxoethyl]- | | 24%/18% (10) |
| 4-methyl-5-(2′-hydroxyethyl)-thiazolium bromide | | |
| 2,3-Diamino-4,5-dimethyl thiazolium mesitylene | | 24%/23% (10) |
| sulfonate | | |
| 2,3-Diamino-4-methyl-5-hydrozyethyl-thiazolium | | 20%/18% (10) |
| mesitylene sulfonate | | |
| 2,3-Diamino-5-(3′,4′-trimethylenedioxy phenyl)- | | 13%/42% (1) |
| thiazolium mesitylene sulfonate | | |
| 3[2-(1′,4′-benzodioxan-6-yl)-2-oxoethyl]-4-methyl-5- | | 11%/21% (3) |
| (2′-hydroxyethyl)-thiazolium bromide | | |
| 3-[2-(3′,4′-trimethylenedioxyphenyl)-2-oxoethyl]- | | 17%/18% (10) |
| thiazolium bromide | | |
| 3-[2-(3′,5′-di-tert-butyl-4′-hydroxyphenyl)-2- | | 14%/2% (0.3) |
| oxoethyl]-4-methyl-thiazolium bromide | | |
| 3-[2-(3′,5′-di-tert-butyl-4′-hydroxyphenyl)-2- | 3/0/74 | 65%/69% (1) |
| oxoethyl]-5-methyl-thiazolium bromide | | |
| 3-[2-(3′,5′-di-tert-butyl-4′-hydroxyphenyl)-2- | | 48%/49% (10) |
| oxoethyl]-5-methyl-thiazolium bromide | | |
| 1-methyl-3-[2[(3′,5′-di-tert-butyl-4′-hydroxyphenyl)-2- | | 56%/38% (10) |
| oxoethyl]-imidazolium bromide | | |
| 3-(2-phenyl-2-oxoethyl)-4-methyl-5-vinyl-thiazolium | ND/0.1 | 62%/82% (1) |
| bromide | | |
| 3-[2-(3′,5′-tert-butyl-4′-hydroxyphenyl)-2-oxoethyl)-4- | ND/0/60% | 32%/50% (0.3) |
| methyl-5-vinyl-thiazolium bromide | | |
| 3-(2-tert-butyl-2-oxoethyl)-thiazolium bromide | | 28%/37% (10) |
| 3-(3′-methoxybenzyl)-4-methyl-5-(2′-hydroxyethyl)- | | 4%/19% (10) |
| thiazolium chloride | | |
| 3-(3′-methoxybenzyl)-4-methyl-5-(2′-hydroxyethyl)- | | 14%/25% (10) |
| thiazolium chloride | | |
| 3-(2′,6′-dichlorobenzyl)-4-methyl-5-(2′-hydroxyethyl)- | | 6%/27% (10) |
| thiazolium chloride | | |
| 3-(2′-nithrobenzyl)-4-methyl-5-(2′-hydroxyethyl)- | | 11%/13% (10) |
| thiazolium bromide | | |

### EXAMPLE 10

To ascertain the ability of the compounds of the invention to decrease the amount of IgG crosslinked to circulating red blood cells in streptozotocin-induced diabetic rats, was measured by the following assay. The test compounds are administered to the test animals either orally or intraperitoneally, and the blood samples are collected are tested at various times, e.g. 4, 7 or 19 days, after administration to assess efficacy. Protocol for RBC-IgG assay

### A. Preparation of Red Blood Cells

Blood is collected from the rats in heparinized tubes and spun at 2000×g for 10 minutes, and the plasma carefully removed. Then, about 5 ml of PBS per ml blood is added, gently mixed, and then spun again. The supernatant is then removed by aspiration. The wash is then repeated two more times. Then, 0.2 to 0.3 ml of packed RBC is withdrawn from the bottom of the tube, using a pipette, and added to the PBS to make a 1 to 10 dilution. This dilution is then further diluted 1 to 25 and 1 to 50 in PBS.

### B. Assay Set Up.

### 1. Warm Superbloc to 37° C.

2. Take a plate of Multiscreen-HA, 0.45 u. Cellulose ester membrane-sealed 96 well plate (Millipore MARAS45).
3. Wet the wells with 100 μl of PBS.
4. Add 300 μl of superblock to each well and incubate at 37° C. for one hour.
5. Place the plate on the Millititer Vacuum holder, turn on the vacuum and press the plate down once for tight hold. The liquids in the wells will be suctioned off. Wash the wells with 300 μl of PBS-Tween 0.05%.
6. Turn off the vacuum and add 100 μl of PBS to each well.
7. Gently vortex the RBC samples and pipette 50 μl to the wells, as per the protocol sheet. Leave first three wells for reagent blanks. Leave another three wells for antibody blank.
8. Suction-off the liquid as above and wash the RBCs twice with PBS.

### 9. Dilute AP(Rb-anti-rat) (Sigma A-6066), 1 to 25000 in PBS.

10. Add 50 μl to the wells and let stand at room temp. for two hours.
11. Suction-off the liquid as above and wash the RBCs twice with PBS.
12. Add pNPP substrate (1 mg/mi in DEA buffer). 100 μl per well.
13. Let the color develop for two hours at 37° C.
14. Place a 96 well coming micrometer plate in the vacuum chamber.
15. Place the sample plate on the vacuum manifold. Make sure the bottom of the plate is completely dry.
16. Apply vacuum for about 5 minutes. Add 100 μl of PBS to all wells and apply vacuum again for 5 minutes. Gently lift the plate and make sure that no liquid drops are hanging at the bottom of the plate. If necessary apply vacuum for few more minutes. Read OD of the solution collected in the Corning plate on Dynatech Plate reader Sample filter 1 and Ref. filter 4.
17. Calculate percent breaking: 100*(OD410 control-OD410 treated)/OD410 control. Percent Inhibition in animals dosed orally at a rate of 10 mg/kg body weight are as listed below:

| | |
|---|---|
| 3-amino-4-methyl-5-vinyl-thiazolium | 11- ± 1 @ 19 days |
| mesitylenesulfonate | |
| 3-[2-(2′-naphthyl)-2-oxoethyl)-4- | 40 ± 24 @ 19 days |
| methyl-5-(2′-hydroxyethyl)-thiazolium | |
| bromide | |
| 3-[2-(3′,5′-di-tert-butyl-4′-hydroxy- | 65 ± 15 @ 19 days |
| phenyl)-2-oxoethyl]-5-methyl-thiazolium | |
| bromide | |
| 3-(2-phenyl-2-oxoethyl)-4-methyl-5- | 58 ± 21 ® 19 days |
| vinyl-thiazolium bromide | |

The extensive degree of reversal of crosslinking observed in these studies underscores two important conclusions by Applicants. First, a large percentage of cross-links formed in vivo are susceptible to attack and cleavage by the dinucleophilic, thiazolium-based compounds of the present invention, and thus, by inference, that these cross-links comprise an α-diketone segment consistent with the model shown in Schemes A and B. Second, the crosslink-breaking compounds of the present invention can act catalytically, in the sense that a single, dinucleophilic thiazolium-based molecule of the present invention can attack and cause the cleavage of more than one glycation cross-link.

### EXAMPLE 11

This example describes the preparation of CNBr peptide maps of rat laid tendon collagen from normal and diabetic animals following treatment with a compound of the invention, i.e., 3-(2-phenyl-2-oxoethyl)thiazolium bromide. Collagen fibers (5 mg) from streptozotocin diabetic rats and age-matched control animals hydrated in land PBS at 60° C. for one hour, the soluble collagen was removed and the pellets were washed several times with PBS then treated with 3-(2-phenyl-2-oxoethyl)thiazolium bromide at a concentration of 30 mM for 16 hours. Following incubation, the pellets were centrifuged, washed, and treated with CNBr (40 mg/ml in formic acid at 30° C. for 48 hours. The CNBr digests were lyophilized repeatedly to remove CNBr and acid and then subjected to SDS-PAGE (20% acrylamide) under reducing conditions (Lanes 1, 2 and 9, MWS; lane 3, 4 and 5, tail tendon collagen from non-diabetic animals with 3 and 5 treated with 3-(2-phenyl-2-oxoethyl) thiazolium bromide, 4 was treated with PBS; lanes 6, 7 and 8, collagen from diabetic animals with 6 and 8 treated with 3-(2-phenyl-2-oxoethyl)thiazolium bromide, 7 was treated with PBS).

### EXAMPLE 12

### Preparation of AGE-BSA and Crosslinked-AGE-BSA

Prepare the following solutions.

| | | |
|---|---|---|
| 1. | Buffer: | 0.4 M sodium phosphate pH 7.4. |
| | NaH₂PO₄: | 6 g/100 ml |
| | NaH₂PO₄: | 7 g/100 ml |

pH of the monobasic sodium phosphate was adjusted to 7.4 with the dibasic 0.02 sodium azide was added per 100 ml of the buffer.

### 2. BSA Solution

BSA: Calbiochem Type V; 400 mg/ml in the buffer 1. Total volume prepared 50 g/125 ml. Filtered through a 0.45 u filter into a sterile one liter Corning flask.
3. Glucose solution. 400 uM
Glucose: 400 mM 9 g/125 ml of buffer. Filtered through a 0.45 u filter into one liter Corning sterile flask.

### Reaction Setup:

BSA and glucose solutions (100 ml each) were mixed in the one liter Corning sterile flask, screw-capped tight and incubated at 56° C. without shaking. The bottle was opened once a week to remove aliquots for testing. Reaction was continued for 9 weeks when AGE-BSA polymer formation was observed.

### Breaking the Polymer:

Pieces of AGE-BSA gel was washed with PBS until no more protein was leached in the supernatant, blotted dry with paper towels. About 50 mg of the washed gel was incubated either with PBS or 10 mm 3-(2-phenyl-2-oxoethyl)thiazolium bromide overnight at 37° C. The supernatants were analyzed by SDS-PAGE and stained with coommassie blue.

### EXAMPLE 13

The cross-link structure and related compounds of the present invention also find utility as antigens or haptens, to elicit antibodies specifically directed thereto. Such antibodies, likewise of the present invention, are useful in turn to identify AAA structures of the present invention. By constructing immunoassays employing anti-cross-link structure antibodies of the present invention, for instance, the degree to which proteins are modified by such cross-links can be measured. As discussed above, and depending on the half-life of the protein so modified, immunochemical measurement of the cross-link epitopes on a protein sample, such as hemoglobin, provides an index of recent AGE-formation. Likewise, immunochemical detection of cross-link epitopes on circulating and/or tissue proteins can be used to monitor the course of therapy with compounds of the present invention, which compounds are directed toward inhibition of, and breaking of advanced glycation.

Cross-link-modified BSA for use as an immunogen can be prepared by coupling a cross-link structure with bovine serum albumin (BSA) using any of a number of well-known divalent coupling reagents such as a carbodiimide like EDC. Various other haptens, antigens, and conjugated immunogens corresponding to the cross-link structures of the present invention, including without limitation those described specifically herein, can conveniently be prepared, either by isolation from incubation mixtures or by direct synthetic approaches. This cross-structure may then be used as an immunogen to raise a variety of antibodies which recognize specific epitopes or molecular features thereof.

In a preferred embodiment, the cross-link structure itself is considered a hapten, which is correspondingly coupled to any of several preferred carrier proteins, including for instance keyhole limpet hemocyanin (KLH), thyroglobulin, and most preferred, bovine serum albumin (BSA), using a divalent coupling reagents such as EDC, according to protocols widely circulated in the art.

The cross-link structure, whether alone or coupled to a carrier protein, may be employed in any well-recognized immunization protocol to generate antibodies and related immunological reagents that are useful in a number of applications owing to the specificity of the antibodies for molecular features of the cross-link structure.

Following a preferred protocol, any of several animal species may be immunized to produce polyclonal antisera directed against the cross-link structure-protein conjugate, including for instance mice, rats, hamsters, goats, rabbits, and chickens. The first of three of the aforesaid animal species are particularly desired choices for the subsequent production of hybridomas secreting hapten-specific monoclonal antibodies. The production of said hybridomas from spleen cells of immunized animals may conveniently be accomplished by any of several protocols popularly practiced in the art, and which describe conditions suitable for immortalization of immunized spleen cells by fusion with an appropriate cell line, e.g. a myeloma cell line. Said protocols for producing hybridomas also provide methods for selecting and cloning immune splenocyte/myeloma cell hybridomas and for identifying hybridomas clones that stably secrete antibodies directed against the desired epitope(s). Animal species such as rabbit and goat are more commonly employed for the generation of polyclonal antisera, but regardless of whether polyclonal antisera or monoclonal antibodies are desired ultimately, the hapten-modified carrier protein typically is initially administered in conjunction with an adjuvant such as Complete Freund's Adjuvant. Immunizations may be administered by any of several routes, typically intraperitoneal, intramuscular or intradermal; certain routes are preferred in the art according to the species to be immunized and the type of antibody ultimately to be produced. Subsequently, booster immunizations are generally administered in conjunction with an adjuvant such as alum or Incomplete Freund's Adjuvant. Booster immunizations are administered at intervals after the initial immunization; generally one month is a suitable interval, with blood samples taken between one and two weeks after each booster immunization. Alternatively, a variety of so-called hyperimmunization schedules, which generally feature booster immunizations spaced closer together in time, are sometimes employed in an effort to produce anti-hapten antibodies preferentially over anti-carrier protein antibodies.

The antibody titers in post-boost blood samples can be compared for hapten-specific immune titer in any of several convenient formats including, for instance, Ouchterlony diffusion gels and direct ELISA protocols. In a typical direct ELISA, a defined antigen is immobilized onto the assay well surface, typically in a 96-well or microtiter plate format, followed by a series of incubations separated by rinses of the assay well surface to remove unbound binding partners. By way of non-limiting example, the wells of an assay plate may receive a dilute, buffered aqueous solution of the hapten/carrier conjugate, preferably wherein the carrier protein differs from that used to immunize the antibody-producing animal to be tested; e.g. serum from AAA/KLH conjugate-immunized animal might be tested against assays wells decorated with immobilized AAA/BSA conjugate. Alternatively, the assay surface may be decorated by incubation with the hapten alone. Generally, the surface of the assay wells is then exposed to a solution of an irrelevant protein, such as casein, to block unoccupied sites on the plastic surfaces. After rinsing with a neutral buffered solution that typically contains salts and a detergent to minimize non-specific interactions, the well is then contacted with one of a serial dilution of the serum prepared from the blood sample of interest (the primary antiserum). After rinsing again, the extent of test antibodies immobilized Onto the assay wells by interaction with the desired hapten or hapten/carrier conjugate can be estimated by incubation with a commercially available enzyme-antibody conjugate, wherein the antibody portion of this secondary conjugate is directed against the species used to produce the primary antiserum; e.g. if the primary antiserum was raised in rabbits, a commercial preparation of anti-rabbit antibodies raised in goat and conjugated to one of several enzymes, such as horseradish peroxidase, can be used as the secondary antibody. Following procedures specified by the manufacturer, the amount of this secondary antibody can then be estimated quantitatively by the activity of the associated conjugate enzyme in a calorimetric assay. Many related ELISA or radioimmunometric protocols, such as competitive ELISAs or sandwich ELISAs, all of which are well know in the art, may optionally be substituted, to identify the desired antisera of high titer; that is, the particular antisera which give a true positive result at high dilution (e.g. greater than 1/1000and more preferably greater than 1/10,000).

Similar immunometric protocols can be used to estimate the titer of antibodies in culture supernatants from hybridomas prepared from spleen cells of immunized animals. In so characterizing antisera or hybridoma supernatants, it is desirable to employ a variety of control incubations, e.g. with different carrier proteins, related but structurally distinct haptens or antigens, and omitting various reagents in the immunometric procedure in order to minimize non-specific signals in the assay and to identify reliable determinations of antibody specificity and titer from false positive and false negative results. The types of control incubations to use in this regard are well known. Also, the same general immunometric protocols subsequently may be employed with the antisera identified by the above procedures to be of high titer and to be directed against specific structural determinants in the cross-link structures on biological samples, foodstuffs or other comestibles, or other amine-bearing substances and biomolecules of interest. Such latter applications of the desired anti-aldehyde-modified Amadori product antibodies, whether polyclonal or monoclonal, together with instructions and optionally with other useful reagents and diluents, including, without limitation, a set of molecular standards of the cross-link structure, may be provided in kit form for the convenience of the operator.

The working examples describing the ability of the disclosed thiazolium compounds to break, or prevent the formation, of AGEs, as described in U.S. Pat. No. 5,656,261; U.S. Pat. No. RE38330; U.S. Pat. No. 5,853,703; U.S. Pat. No. 6,007,865; U.S. Pat. No. 6,440,749; U.S. Pat. No. 7,022,719; U.S. Pat. No. 7,166,625; U.S. Pat. No. 7,166,625; U.S. Patent Publication No. 2002/0192842; U.S. Patent Publication No. 2007/0025926; U.S. Patent Publication No. 2002/0068729; U.S. Patent Publication No. 2003/0176426; U.S. Patent Publication No. 2007/0043016 and U.S. Patent Publication No. 2004/0235837 are incorporated by reference in there entireties.

The following section below entitled Materials and Methods applies to Examples 14-21:

### Materials and Methods

### Animals.

Adult (8-week-old) male C57BL6JJ mice (Precinct Animal Centre, Melbourne, Australia) and RAGE knockout (KO) mice¹⁷ (Liliensiek et al., J Clin Invest. 113:1641-50, 2004) were housed in a temperature-controlled room on a 12-h light-dark cycle. Mice were placed on either a standard diet (AIN-93G; Specialty feeds, Perth AUS; n=10/group) or a high fat (HF) diet formulated to mimic a “Western fast food diet”, (SF05-031, Specialty Feeds; n=10). Both diets contained the same vitamin and calorie content. However, 21% of total energy in the SF05-031 diet was derived from fat (vs. 7% in AIN-93G), based on the addition of clarified butter (Ghee; 210 g/kg).

The SF05-031 diet also contained elevated levels of AGEs, including a 6-fold increase in the content of the AGE, carboxymethyllysine (CML), determined by GC-mass spectroscopy. Animals receiving a HF diet were further randomized to receive alagebrium chloride at a dose of 1 mg/kg/day delivered by oral gavage. All groups were followed for 16 weeks.

### Measurement of Physiological and Biochemical Parameters.

The following parameters were serially measured in all groups: body weight; blood glucose, measured using a glucometer; systolic blood pressure, measured by tail-cuff plethysmography in conscious, warmed mice.

### Assessment of Cardiac Hypertrophy

The total cardiac and isolated left ventricular mass was measured at sacrifice, and expressed adjusted for body surface area in square meters (m²). Cardiomyocyte hypertrophy was assessed by measuring cross-sectional area of 100 cardiomyocytes in the left ventricle near the endocardial region, assessing those with nearly circular capillary profiles.

Cardiac hypertrophy in obesity reactivates the fetal gene program, characterized by a switch to β-Myosin heavy chain (β-MHC) expression in the adult murine heart⁸ (Ng et al., Circ Res. 68:1742-50, 1991). Gene expression of markers of pathological hypertrophy, β-MHC and α-MHC were assessed by real-time quantitative RT-PCR. This was performed using the TaqMan system based on real-time detection of accumulated fluorescence, as previously described¹⁹ (Thomas et al., J Am Soc Nephrol. 16:2976-84, 2005). Gene expression was normalized to 18S mRNA and reported as ratios compared to the level of expression in untreated control mice, which were given the arbitrary value of one.

### Assessment of Myocardial Fibrosis

Previous studies have shown that obesity is associated with an increase in intramyocardial fibrosis. Gene expression of markers of fibrosis, including collagens and fibronectin were assessed by real-time quantitative RT-PCR, as detailed above. Perivascular collagen deposition was assessed by Van Giesen's Stain and interstitial fibrosis was assessed by staining with Picrosirus red staining of organized collagen. stained area of interstitial to total area of cardiac tissue in each section. Paraffin-fixed cardiac sections were also stained for collagen IV by immunohistochemistry, as previously described¹² (Candido et al., Circ Res. 92:785-92, 2003).

### Assessment of Myocardial Inflammation

The expression of inflammatory cytokines, tumor necrosis factor (TNFα), interleukin-6 (IL-6), along with macrophage infiltration, is augmented following cardiac stress. The gene expression of IL-6, TNF α, the adhesion molecule, ICAM-1 and the chemokine, macrophage chemoattactant factor (MCP-1) in left ventricular homogenates were assessed by real-time quantitative RT-PCR. The myocardial expression of IL-6 protein was further quantitated by ELISA.

### Assessment of Superoxide Production

The production and accumulation of superoxide and other ROS is a key mediator of carida injury and cellular dysfunction associated with high fat feeding⁵ (Boudina and Abel, Circulation 115:3213-23, 2007). To assess superoxide production in fat fed mice, left ventricles were rapidly excised, and homogenised in oxygen-saturated Krebs buffer (containing 118 mM NaCl, 4.7 mM KCl, 1.2 mM MgSO₄.7H₂O, 1.2 mM KH₂PO₄, 11 mM D-glucose, 0.03 mM EDTA and 2.5 mM CaCl₂, pH 7.4). Fresh homogenates were then divided to determine superoxide production in the presence of 125 μM NADH and 30 μM of the NADPH oxidase inhibitor, diphenylene iodinium (designated NADH-dependent superoxide production), and superoxide production in the presence of 125 μM NADPH and 30 μM of the respiratory chain inhibitor, rotenone (designated NADPH-dependent superoxide production). This latter activity was not inhibited by L-N^{G}-Nitroarginine or allopurinol. Each of the samples was processed in triplicate. Following incubation for 60 min at 37° C., before the rate of superoxide anion formation was determined by the addition of 3.8 μM lucigenin (bis-N-methylacridinium nitrate). Chemiluminescence was monitored every 6 min for 60 min, and the integral over this period was expressed as relative light units (RLUs) normalised to 10 mg tissue weight. The expression of the NADPH subunits, NOX-2 (gp91^{phox}), p47^{phox}, NOX-4 and RAC-1 in left ventricular homogenates, along with the expression of heme oxygenase (HO) and glutathione peroxidase (GPX), key defence mechanism against oxidative stress, were further assessed by real-time quantitative RT-PCR, as detailed above.

### Assessment of Lipid Metabolism

Changes in myocardial energy metabolism, due to altered substrate handling, characterize obesity-related cardiac disease⁵ (Boudina and Abel, Circulation 115:3213-23, 2007). In particular, intra-myocardial accumulation of triacylglycerol metabolites mediated by activation of peroxisome proliferator activated receptor α(PPARα) has been strongly associated with cardiac dysfunction in rodents fed a HF diet²⁰ (Ouwens et al., Diabetologia 48:1229-37, 2005). To assess the interaction of AGE/RAGE with lipotoxicity pathways in this model, the ventricular expression of PPARα and the fatty acid translocase, CD36, were assessed by real-time quantitative RT-PCR. Intra-myocardial accumulation of triacylglycerol metabolites was assessed by staining with Oil red-O in frozen cardiac sections and quantitated in cardiac homogenates extracted using the method of Bligh and Dyer and measured using a standard commercial enzymatic assay.

### Accumulation of RAGE Ligands and AGE Receptor Expression

Cardiac levels of AGEs were estimated using a indirect in-house ELISA, as previously described²¹ (Coughlan et al., Endocrinology 148:886-95, 2007), with a monoclonal AGE antibody that recognizes the non-fluorescent AGE, carboxymethyl-lysine (CML) at its primary epitope. The expression of non-AGE RAGE ligand, S100 A8/A9 (calprotectin, MRP 8/14)²² (Ehlermann et al. Cardiovasc Diabetol. 5:6, 2006) was also assessed by commercial ELISA. The expression of RAGE, together with the AGE clearance receptors AGE-R1 and AGE-R3 were further assessed by real-time quantitative RT-PCR.

### Statistical Analysis.

Continuous data are expressed as mean±SEM except where otherwise specified. Differences in continuous variables were compared using Student's t tests (2 groups) or one-way ANOVA (3 or more groups). Spearman rank order correlation was used to analyse associations between continuous variables. Differences in categorical variables were compared using the Mann-Whitney rank sum test. A p value of <0.05 was considered statistically significant.

### EXAMPLE 14

### Weight and Blood Glucose Changes

Feeding with a HF diet resulted in a significant increase in body weight, in both C57BL6JJ and RAGE KO mice, when compared to animals fed a healthy diet (Table 1). Treatment with alagebrium chloride, reduced weight-gain associated with a western diet, but did not return it to control levels. This was not due to differences in food intake, which were unaffected by the addition of alagebrium chloride. Blood glucose were also significantly increased with a HF diet, with equivalent changes seen in both C57BL6 and RAGE KO mice and animals treated with alagebrium (Table 1). Fifteen weeks of HF feeding did not influence systolic blood pressure levels in C57BL6 or RAGE KO mice, and the systolic blood pressure was not modified in mice treated with alagebrium chloride.

TABLE 1

| | Wild-type | | | RAGE KO | | |
|---|---|---|---|---|---|---|
| | SD | HF | HF + AL | SD | HF | HF + AL |
| Weight gain (% baseline) | 10 ± 3 | 40 ± 3* | 27 ± 2*^{#} | 35 ± 3* | 64 ± 7*^{#} | 44 ± 7* |
| Glucose (mmol/L) | 13.4 ± 0.3 | 16.3 ± 0.3* | 15.1 ± 0.5*^{#} | 12.9 ± 0.2 | 15.9 ± 0.5* | 13.7 ± 0.7^{#} |
| Total cardiac mass (g/m²) | 14.9 ± 0.7 | 13.8 ± 0.6 | 14.5 ± 0.4 | 14.8 ± 0.5 | 14.3 ± 0.6 | 13.7 ± 0.6 |
| Total LV mass (g/m²) | 9.4 ± 0.5 | 8.5 ± 0.5 | 9.5 ± 0.3 | 10.0 ± 0.6 | 10.0 ± 0.5 | 9.7 ± 0.3 |
| Mean myocyte diameter (μm) | 16.8 ± 0.5 | 20.5 ± 0.8* | 19.7 ± 0.8* | 17.0 ± 0.6 | 19.0 ± 0.5* | 19.5 ± 0.6* |

### EXAMPLE 15

### Cardiac Hypertrophy in HF-fed Mice

There was no significant change in the surface area adjusted total cardiac or isolated left ventricular mass in C57BL6 or RAGE KO mice following 15-weeks of feeding with a HF diet (Table 1). However, the cross-sectional area of cardiomyocytes in the left ventricular free wall was significantly increased following feeding with a HF diet in C57BL6 KO mice (Table 1). In addition, the cardiac expression of β-Myosin heavy chain was elevated following 15-weeks of HF feeding in C57BL6, while the expression of α-Myosin heavy chain was unaltered (Table 2). Table 2 shows the expression of mediators of cardiac dysfunction associated with a high fat diet, in C57b16 and RAGE KO mice, measured by real time RT-PCR. HF feeding RAGE KO mice failed to induce cardiac hypertrophy (Table 1) or increase the expression of β-Myosin heavy chain gene (Table 2). Treatment with alagebrium chloride had no effect on hypertrophy or the expression of β-Myosin heavy chain gene in HF-fed animals, but reduced the expression of α-Myosin heavy chain in both wild type and RAGE KO animals.

TABLE 2

| Gene expression | Wild-type | | | RAGE KO | | |
|---|---|---|---|---|---|---|
| (AU/18s) | SD | HF | HF + AL | SD | HF | HF + AL |
| RAGE (%) | 1.0 ± 0.2 | 2.5 ± 0.4* | 1.0 ± 0.2^{#} | n.d | n.d | n.d |
| Hypertrophy: | | | | | | |
| Alpha MHC (%) | 1.0 ± 0.2 | 1.0 ± 0.1 | 0.7 ± 0.2* | 0.8 ± 0.1 | 1.0 ± 0.2 | 0.6 ± 0.2*^{#} |
| Beta MHC (%) | 1.0 ± 0.2 | 3.2 ± 0.5* | 2.5 ± 0.3* | 0.9 ± 0.2 | 1.3 ± 0.3^{#} | 1.6 ± 0.2*^{#} |
| Inflammation: | | | | | | |
| Interleukin-6 (%) | 1.0 ± 0.3 | 18.0 ± 5.3* | 0.2 ± 0.1*^{#} | 0.9 ± 0.2 | 0.3 ± 0.1*^{#} | 0.4 ± 0.1*^{#} |
| TNF-alpha (%) | 1.0 ± 0.2 | 2.0 ± 0.3* | 0.5 ± 0.2*^{#} | 1.4 ± 0.3 | 0.3 ± 0.1*^{#} | 0.4 ± 0.1*^{#} |
| ICAM-1 (%) | 1.0 ± 0.1 | 5.6 ± 0.8* | 0.2 ± 0.1*^{#} | 1.0 ± 0.2 | 0.6 ± 0.1*^{#} | 0.2 ± 0.1*^{#} |
| MCP-1 (%) | 1.0 ± 0.1 | 3.0 ± 0.6* | 0.4 ± 0.1*^{#} | 1.0 ± 0.1 | 0.7 ± 0.2*^{#} | 0.2 ± 0.1*^{#} |
| P-65 (%) | 1.0 ± 0.1 | 1.5 ± 0.2* | 0.8 ± 0.2*^{#} | 0.5 ± 0.1* | 0.5 ± 0.1*^{#} | 0.3 ± 0.1*^{#} |
| Fibrosis: | | | | | | |
| Collagen I (%) | 1.0 ± 0.1 | 1.6 ± 0.1* | 1.6 ± 0.1* | 0.4 ± 0.1* | 0.7 ± 0.1*^{#} | 0.8 ± 0.1^{#} |
| Collagen IV (%) | 1.0 ± 0.1 | 1.3 ± 0.1* | 1.3 ± 0.1* | 0.6 ± 0.1* | 0.7 ± 0.1*^{#} | 0.9 ± 0.1^{#} |
| Oxidative stress: | | | | | | |
| gp91^{phox} (%) | 1.0 ± 0.1 | 1.8 ± 0.3* | 0.8 ± 0.2^{#} | 1.0 ± 0.1 | 1.0 ± 0.1 | 0.9 ± 0.1 |
| RAC-1 (%) | 1.0 ± 0.1 | 1.0 ± 0.1 | 1.0 ± 0.2 | 0.7 ± 0.1* | 0.7 ± 0.1* | 0.7 ± 0.1* |
| Heme oxygenase (%) | 1.0 ± 0.2 | 0.2 ± 0.1* | 0.1 ± 0.1* | 1.0 ± 0.2 | 0.2 ± 0.1* | 0.1 ± 0.1* |
| Metabolic: | | | | | | |
| PPAR alpha (%) | 1.0 ± 0.2 | 3.0 ± 0.5* | 1.9 ± 0.4*^{#} | 1.0 ± 0.2 | 3.0 ± 0.6* | 1.2 ± 0.3^{#} |

### EXAMPLE 16

### Myocardial Fibrosis in HF-Fed Mice

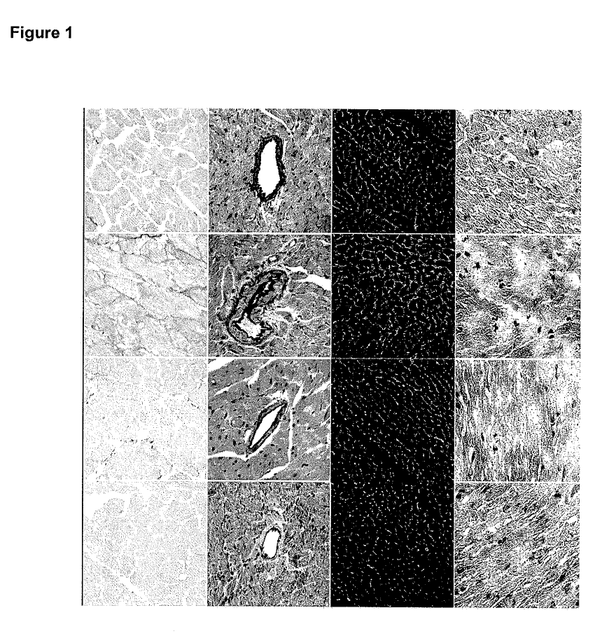

Previous studies have shown that diabetes is associated with an increase in intramyocardial fibrosis, that can be prevented by inhibition of AGE accumulation¹² (Candido et al., Circ Res. 92:785-92, 2003). Feeding a HF diet also resulted in a significant increase in the gene expression of collagen I and collagen IV in c57B16 mice (Table 2). Untreated RAGE KO mice had lower baseline level of collagen I and collagen IV gene expression than untreated c57 controls. However, HF feeding in these animals failed to significantly increase cardiac collagen expression beyond that seen in animal receiving a normal diet (Table 2). The differences were confirmed with significant interstitial collagen deposition in HF fed mice (FIG. 1), which was reduced in RAGE KO animals fed the same diet (FIG. 1). However, collagen deposition was unaffected by treatment with alagebrium chloride in HF-fed animals.

### EXAMPLE 17

### Myocardial Inflammation in HF-fed Mice

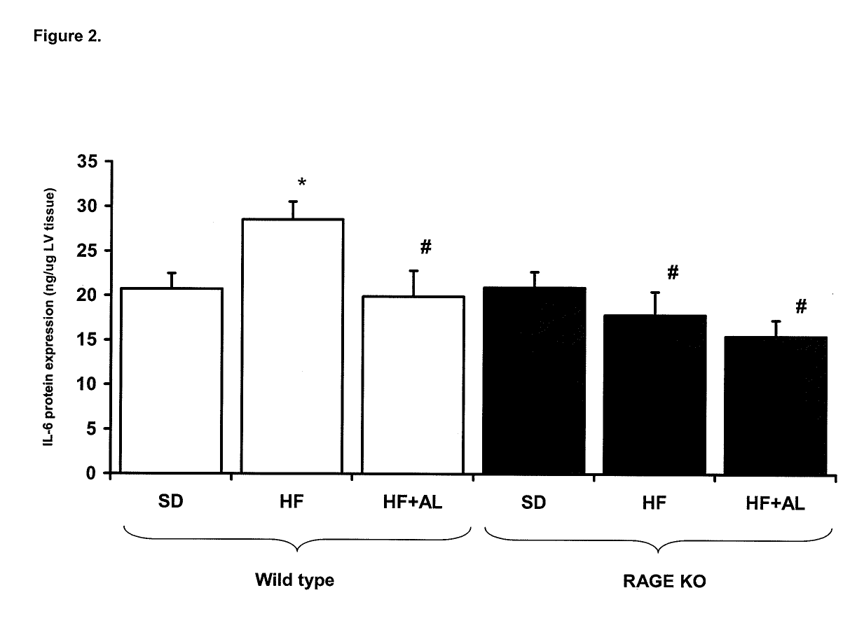

Myocardial inflammation is significantly increased in animals fed a HF diet¹ (Aguila et al., Mech Ageing Dev. 122:77-88, 2001). Fifteen weeks of HF feeding resulted in a significant increase in the gene expression of IL-6 and TNF α in c57B16 mice (Table 2). This was associated with a significant increase in myocardial IL-6 expression at a protein level (FIG. 2). Untreated RAGE KO mice had reduced expression of inflammatory cytokines, when compared to untreated c57B16 controls (Table 2, FIG. 2). In addition, HF feeding in RAGE KO animals failed to induce cardiac inflammation. Indeed, there was a paradoxical decline in the expression of these inflammatory cytokines following HF feeding when compared to RAGE KO mice receiving a normal (low AGE) diet. The increase in these cytokines in wild type fed a HF diet was also reduced by alagebrium chloride, although this agent had no significant effect in RAGE KO mice.

Cardiac expression of the macrophage chemo-attractant protein (MCP-1) and the intracellular adhesion molecule (ICAM-1) were also increased in c57B16 mice fed a high fat diet. Following the same trend observed for inflammatory cytokines, the expression of both ICAM-1 and MCP-1 were significantly reduced in RAGE KO mice (Table 2). Treatment with the AGE inhibitor, alagebrium chloride, also reduced the expression of ICAM-1 and MCP-1 in both wild type and RAGE KO mice.

It is known that activation of RAGE triggers the activation of inflammatory pathways via NF-κB. In this study, the HF feeding significantly increased the expression of NF-κBp65 mRNA (Table 2). This increase was prevented in animals treated with alagebirum chloride. Signaling through NF-κB dependent pathways was marked down-regulated in RAGE KO mice, consistent with previous reports in these animals¹⁷ (Liliensiek et al., J Clin Invest. 113:1641-50, 2004).

### EXAMPLE 18

### Myocardial AGE Accumulation and Receptor Expression in HF-fed Mice

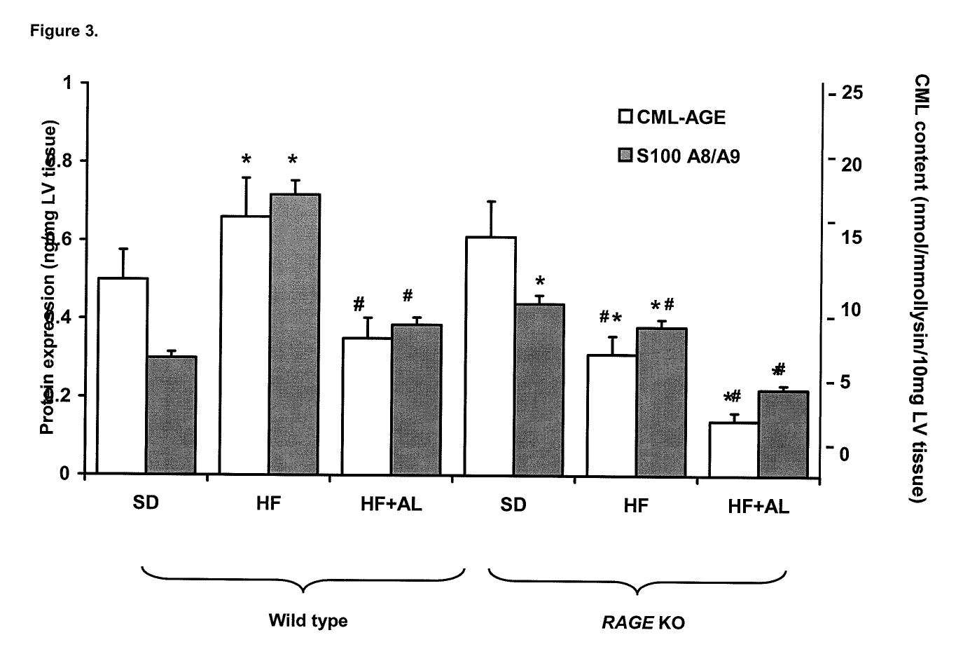

HF feeding resulted in a small, but significant increase in AGE accumulation, as demonstrated by ELISA for CML-modified protein (FIG. 3). RAGE KO mice fed a standard diet had a similar level of cardiac CML-AGE to wild type animals. However, HF feeding in RAGE KO mice was associated with a paradoxical reduction in the AGE content of cardiac tissues. AGE accumulation was also markedly reduced following treatment with the AGE inhibitor, alagebrium chloride, in both wild type and RAGE KO mice.

HF feeding was also associated with a significant accumulation in the non-AGE RAGE ligand, S100 A8/A9 (FIG. 3), correlating with changes in other inflammatory cytokines, as detailed above. This increase in S100 A8/A9 following HF feeding, was also attenuated in RAGE KO mice and in animals receiving alagebrium chloride. Interestingly, basal levels of S100 A8/A9 were increased in the RAGE KO mouse when compared to wild type animals receiving a normal diet (FIG. 3).

Paralleling changes in RAGE ligands, the gene and protein expression of RAGE was significantly increased in HF fed c57BL6 mice when compared to mice fed a standard diet (Table 2). This increase in RAGE expression was prevented in c57BL6 mice treated with alagebrium chloride (Table 2). There was no detectable RAGE expression in RAGE KO mice. Neither a HF diet nor alagebrium influenced the cardiac expression of the AGE clearance receptors, AGE-R1 and AGE-R3, and there was no difference in the cardiac expression of AGE-R1 between RAGE KO mice and C57B16JJ mice.

### EXAMPLE 19

### Superoxide Production in HF-fed Mice

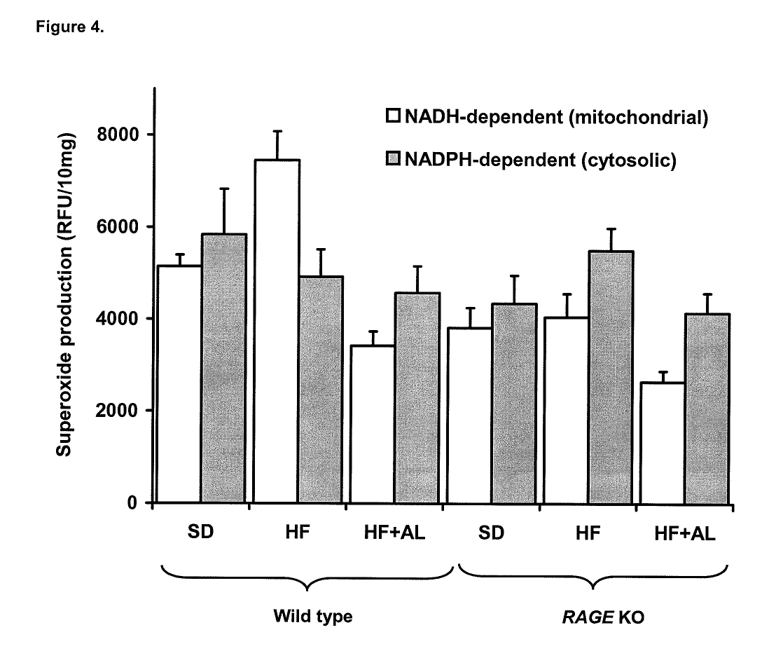

HF feeding was associated with a significant increase in NADH-dependent (mitochondrial) superoxide production in cardiac homogenates from wild type mice (FIG. 4). However, high fat feeding in RAGE KO failed to increase mitochondrial superoxide production in cardiac homogenates. Treatment with the AGE inhibitor, alagebrium chloride, also significantly reduced mitochondrial superoxide production in both wild type and RAGE KO mice.

NADPH-dependent superoxide production was not detectably altered by a HF diet or in RAGE KO mice (FIG. 4). However, cardiac expression of the NADPH oxidase subunit gp91^{phox (NOX-}2) was increased in wild type mice fed a high fat diet. This increase was not observed in RAGE KO animals or in animals treated with alagebrium chloride. Expression of the other NAPHH subunits p47 ^{phox}, Nox-4, and rac-1 were not altered by high fat feeding. However, cardiac expression of the rac-1 gene was lower in RAGE KO mice (Table 2).

A HF diet also resulted in a decline in the expression of heme oxygenase (HO-1), a key protector against super-oxide dependent injury in the heart ²³ (Abraham and Kappas, Free Radic Biol Med. 39:1-25, 2005) Interestingly, this decline was observed in both c57BL6 and RAGE KO mice, and HO-1 expression was not influenced by treatment with alagebrium chloride (Table 2). The expression of GPX-1 and GPX-3 were not affected by feeding with a HF diet.

### EXAMPLE 20

### Cardiac Lipid Metabolism in HF-Fed Mice

HF feeding was associated with the intramyocardial accumulation of lipid particles, as demonstrated by Oil-red O staining (FIG. 1) and on cardiac triglyceride content (standard diet, <0.5 μmol/100 mg LV protein; HF, 8±2 μmol/100 mg LV protein, p<0.01). This increase was similar in wild type and RAGE KO mice and unaffected by treatment with alagebrium chloride. Similarly, the fat-dependent induction of the α-type peroxisome proliferator activated receptor (PPARα) was similar in wild type and RAGE KO mice fed a HF diet (Table 2). However, treatment with the AGE inhibitor, alagebrium chloride reduced the expression of PPARα in both wild type and RAGE KO animals, without modifying fat accumulation. Expression of the fatty acid translocase, CD36, was not altered by HF feeding, and was similar in wild type and RAGE KO mice and following treatment with alagebrium.

### EXAMPLE 21

### Cardiac Disorders and High Fat Feeding

The Western diet has a range of adverse effects in the heart, including inflammation, hypertrophy¹ (Aguila et al., Mech Ageing Dev. 122:77-88, 2001), fibrosis¹ (Aguila et al., Mech Ageing Dev. 122:77-88, 2001) and contractile dysfunction^{2,3} (Wilson et al., Biochem J. 2007; Ouwens et al., Diabetologia 2007). Such actions have largely been ascribed to the fat content of such diets. However, processed diets that are high in fat are also high in AGEs⁷ (Uribarri et al., Ann N Y Acad. Sci. 1043:461-6, 2005). The present invention provides that inhibition of AGE accumulation following treatment with thiazolium compounds, such as alagebrium chloride, or prevention of RAGE activation in RAGE knockout animals prevents the induction of inflammation, oxidative stress and mitochondrial dysfunction in the heart associated with high fat feeding.

Research on AGEs has largely focused on their pathogenic role in the development and progression of diabetic complications, as chronic hyperglycaemia leads to augmented AGE accumulation in vivo (Brownlee, Nature. 414:813-20, 2001). However, AGEs are able to induce end-organ damage, in the absence of hyperglycaemia^{19,22}, (Thomas et al., J Am Soc Nephrol. 16:2976-84, 2005 and Ehlermann et al., Cardiovasc Diabetol. 5:6, 2006) and, indeed, inhibitors of AGE accumulation are able to prevent organ injury in diabetes without normalizing glucose levels¹² (Candido et al., Circ Res. 92:785-92, 2003). Consequently, other states in which AGEs are elevated may also see the development of ‘diabetes-like’ AGE-induced injury, as well as being able to benefit from inhibition of the AGE/RAGE axis. For example, inhibition of cardiac AGE accumulation in the ageing heart reduces myocardial damage⁹⁻¹³ (Liu et al., Am J Physiol Heart Circ Physiol. 285:H2587-91, 2003; Ceylan-Isik et al., J Appl Physiol. 100:150-6, 2006; Corman et al., Proc Natl Acad Sci USA. 95:1301-6, 1998; Candido et al., Circ Res. 92:785-92, 2003; Norton et al., Circulation. 93:1905-12, 1996). The present invention provides compositions and methods for the inhibition of AGE accumulation and/or signalling via the RAGE receptor, which can treat or prevent myocardial inflammation, hypertrophy and oxidative stress associated with patients subjected to a high fat diet, another state associated with high exposure to AGEs.

Activation of RAGE by AGEs and other non-RAGE ligands triggers the activation of secondary messenger pathways, such as NF-κB and oxidative stress, This mechanism has been strongly implicated in the development and progression of cardiac disease. The present invention provides that a diet high in fat (and AGEs) produced sustained NF-κB activation and upregulation of NF-κBp65 and I-κB. Reactive oxygen species produced by the mitochondrial respiratory chain have been described as one major mediator of AGE and hyperglycemia-dependent NF-κB activation²³ (Abraham and Kappas, Free Radic Biol Med. 39:1-25, 2005). The present invention provides that a diet high in fat (and AGEs) produced significant increases in mitochondrial superoxide production in the heart. This increase was prevented in animals treated with thiazolium AGE inhibitors or in RAGE KO animals. While studies in endothelial²⁴ (Brownlee, Nature. 414:813-20, 2001) and smooth muscle cells²⁵ (Robinet et al., Fundam Clin Pharmacol. 21:35-43, 2007) and in the kidney²⁶ (Nishikawa et al., Nature. 404:787-90, 2000) have demonstrated that activation of RAGE by AGEs stimulates NADPH oxidase activity, no detectable change in NADPH-dependent oxidase activity was observed while subjected to a high fat diet. However, cardiac expression of the NADPH oxidase subunit gp91^{phox} (NOX-2) was increased in subjects with a high fat diet. The increase in NOX-2 was normalised following treatment with thiazolium AGE inhibitors, such as alagebrium chloride, and in RAGE KO animals. This indicates that the AGE/RAGE axis also impacts components of the NADPH oxidase pathway in the heart.

The present invention provides that a high fat diet results in a significant increase in various markers of cardiac inflammation, including IL-6, TNFα and ICAM-1, as described in Table 2. These increases were prevented in RAGE KO animals and by treatment with thiazolium AGE inhibitors, such as alagebrium chloride. One possible explanation for this prevention of marker increases in RAGE KO animals may be unopposed activation of anti-inflammatory pathways by AGEs in RAGE KO mice. For example, it is known that AGEs are able to bind and activate a number of different receptors other than RAGE, including AGE-R1 (p60), AGE-R3 (galectin-3)²⁷ (Wautier et al., Am J Physiol Endocrinol Metab. 280:E685-94, 2001) and lysozyme, all of which have anti-inflammatory properties. While the cardiac expression of these AGE receptors was not altered in RAGE KO animals, their preferential activation in the setting of RAGE deficiency, may have contributed to observed anti-inflammatory actions of a HF diet. Cardiac inflammation may also have attenuated by the reduction in AGE levels observed in HF fed RAGE KO mice as shown in FIG. 3. This finding possibly reflects the activation of AGE clearance pathways in other tissues, such as the kidney.

The cardiac consequences of increasing fat intake cannot be fully attributed to AGEs. RAGE KO mice also gained weight, exhibited dysglycemia as shown in Table 1, accumulated intramyocardial fat as shown in FIG. 1 and activated metabolic pathways such as PPARα, to a similar extent as observed in wild type animals fed a HF diet. Similarly, reduced expression of a key cardioproective enzyme³¹ (Kruger et al., J Pharmacol Exp Ther. 319:1144-52, 2006), HO-1 following HF feeding was not affected by strategies to inhibit the AGE/RAGE axis. Such data suggest that the actions of AGEs and RAGE in this model may be partly independent of those of fat, or possible downstream of the metabolic changes associated with increased fat intake.

We claim:

1. A method of treating, ameliorating, or preventing a condition or disorder in a patient subjected to a high fat diet, said condition or disorder selected from the group consisting of a cardiac disorder, myocardial inflammation, myocardial oxidative stress, myocardial AGE accumulation, mitochondrial superoxide production in cardiac cells, RAGE expression or α-type peroxisome proliferator activated receptor (PPARα) expression in cardiac tissue, and weight gain, by administering to a patient in need thereof, a pharmaceutical composition comprising a compound of Formula I, 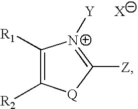
wherein:
R¹ and R² are independently selected from hydrogen, C₁₋₆ linear or branched alkyl and cycloalkyl; or together with their ring carbons form a C₅-C₇ fused cycloalkyl ring having up to two double bonds including any fused double bond of the -olium containing ring, which cycloalkyl ring is optionally substituted by one or more substituents selected from alkyl and fluoro;
Z is hydrogen or C₁₋₆ linear or branched alkyl;
Y is a group of the formula —CH(R⁵)—C(O)—R⁶ wherein
R⁵ is hydrogen, C₁₋₆ linear- or branched-alkyl, or cycloalkyl; and
R⁶ is a C₆ or C₁₀ aryl, wherein R⁶ is optionally substituted with one or more substituents selected from the group consisting of alkyl and halo;
Q is O or S; and
X is a pharmaceutically acceptable anion,
and a pharmaceutically acceptable carrier, thereby treating or preventing said condition or disorder.

2. The method of claim 1 wherein said disorder is a cardiac disorder.

3. The method of claim 2 wherein said cardiac disorder is associated with a high fat diet.

4. The method of claim 1 wherein said disorder is myocardial inflammation.

5. The method of claim 4 wherein said myocardial inflammation is associated with a high fat diet.

6. The method of claim 1 wherein said disorder is myocardial oxidative stress.

7. The method of claim 6 wherein said myocardial oxidative stress is associated with a high fat diet.

8. The method of claim 1 wherein said disorder is myocardial AGE accumulation

9. The method of claim 8 wherein said myocardial AGE accumulation is associated with a high fat diet.

10. The method of claim 1 wherein said disorder is mitochondrial superoxide production in cardiac cells.

11. The method of claim 10 wherein said mitochondrial superoxide production in cardiac cells is associated with a high fat diet.

12. The method of claim 1 wherein said disorder is RAGE expression or α-type peroxisome proliferator activated receptor (PPARα) expression in cardiac tissue.

13. The method of claim 12 wherein said RAGE expression or α-type peroxisome proliferator activated receptor (PPARα) expression in cardiac tissue is associated with a high fat diet.

14. The method of claim 1 wherein said disorder is weight gain.

15. The method of claim 14 wherein said weight gain is associated with a high fat diet.

16. The method of claim 1, wherein R1 and R2 are independently C₁₋₆ linear or branched alkyl.

17. The method of claim 1, wherein Z is hydrogen.

18. The method of claim 1, wherein R⁵ is hydrogen.

19. The method of claim 1, wherein R⁶ is C₆ aryl.

20. The method of claim 1, wherein Q is S.

21. The method of claim 1, wherein the compound of Formula I is 3-(2-phenyl-2-oxoethyl)-4,5-dimethylthiazolium.

22. The method of claim 21, wherein the compound of Formula I is 3-(2-phenyl-2-oxoethyl)-4,5-dimethylthiazolium chloride or 3-(2-phenyl-2-oxoethyl)-4,5-dimethylthiazolium bromide.

23. The method of claim 1, wherein said high fat diet derives greater than about 20% of its total calories from fat.

24. The method of claim 23, wherein said high fat diet derives greater than about 30% of its total calories from fat.

25. The method of claim 23, wherein said high fat diet derives greater than about 40% of its total calories from fat.

26. The method of claim 1, wherein said disorder is not the result of diabetes or adverse sequelae of diabetes, of aging or an age related disorder or of insulin deficiency.

27. The method of claim 1, further comprising administering a modulator of a receptor for advanced glycation end-products (RAGE).

28. The method of claim 1, wherein the myocardial inflammation results in increased cardiac expression of macrophage chemo-attractant protein (MCP-1) or increased cardiac expression of the intracellular adhesion molecule (ICAM-1).

29. The method of claim 1, wherein the mitochondrial superoxide production results in increased cardiac expression of the NADPH oxidase subunit gp91^{phox} (NOX-2).
